(19) United States
(12) Patent Application Publication
(10) Pub. No.: US 2026/0216515 A1
(43) Pub. Date: Jul. 30, 2026

(54) GUIDING THERAPY FOR A PATIENT METRIC

(71) Applicant: INSPIRE MEDICAL SYSTEMS, INC., Golden Valley, MN (US)

(72) Inventors: Maxwell P. Lundeen, Minneapolis, MN (US); Kent Lee, Minneapolis, MN (US)

(73) Assignee: INSPIRE MEDICAL SYSTEMS, INC., Golden Valley, MN (US)

(21) Appl. No.: 19/143,070

(22) PCT Filed: Dec. 20, 2023
(86) PCT No.: PCT/US2023/085232

§ 371 Date: Jun. 25, 2025

Related U.S. Application Data
(60) Provisional application No. 63/477,716, filed on Dec. 29, 2022.

(51) Int. Cl. A61N 1/36

(52) U.S. Cl. CPC A61N 1/36139; A61N 1/3611; A61N 1/36132; A61N 1/36146

(57) ABSTRACT

A device and/or method to guide therapy for a patient metric.

### BACKGROUND

A significant portion of the population suffers from various forms of physiologic conditions, some of which may involve sleep disordered breathing (SDB), related conditions, and/or unrelated conditions. Any therapies aimed at treating such conditions often face challenges in selecting an appropriate level of treatment.

### BRIEF DESCRIPTION OF THE DRAWINGS

FIG. 1A is a diagram schematically representing an example method of determining sleep detection eligibility based on sensed physiologic information.

FIG. 1B is a diagram including a front view schematically representing a patient's body, implantable components, and/or external elements of example methods and/or example devices.

FIG. 1C is a schematic diagram of a control portion.

FIG. 2 is a graph of an example of values of a stimulation energy parameter over a time period.

FIG. 3 is a flow chart of a method and/or device according to an example of the disclosure.

FIG. 4 is a graph of an example change in disease burden and usage over change in stimulation energy parameter.

FIG. 5 is a graph of an example of disease burden and usage over change in values of a stimulation energy parameter.

FIG. 6A is a block diagram of an example stimulation generating portion.

FIGS. 6B-6E are graphs illustrating example patterns of patients titrating stimulation amplitude, such as during an initial use period.

FIG. 6F illustrates an example method for identifying a target location of an infrahyoid muscle-related tissue, such as an infrahyoid muscle-innervating nerve.

FIG. 7 is a block diagram schematically representing an example sensing portion of an example device and/or used as part of example method.

FIG. 8A is a block diagram schematically representing an example stimulation portion.

FIG. 8B is a diagram illustrating example sensing protocol(s) and/or stimulation protocol(s).

FIG. 9A is a block diagram schematically representing an example care engine.

FIGS. 9B and 9C are each a block diagram schematically representing an example control portion.

FIG. 9D is a block diagram schematically representing an example user interface.

FIG. 9E is a diagram schematically representing an example arrangement of communication between a medical device and various example external devices.

FIGS. 9F-9G are flow diagrams schematically representing example methods relating to titration.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to devices for diagnosis, therapy, and/or other care of medical conditions, which may include neural therapy. At least some examples may comprise implantable devices and/or methods comprising use of implantable devices. However, in some examples, the methods and/or devices may comprise at least some external components. In some examples, a therapeutic medical device may comprise a combination of implantable components and external components.

At least some examples of the disclosure include methods comprising guiding therapy for a patient metric. Various examples include a device comprising a control portion programmed to guide therapy for a patient metric. In some examples, the therapy may comprise a stimulation therapy.

Generally, examples of the disclosure evaluate variable stimulation therapy settings of a medical device (MD) (e.g. implantable (IMD)) and patient therapy outcomes, which may include usage, over a period of over one day (i.e. more than one 24 hour period including more than one daily or nightly sleep treatment period) to identify a solution setting of the MD to maximize patient usage of the IMD stimulation therapy while minimizing disease burden (e.g. adverse patient therapy outcome(s)). In some examples, the disease burden may comprise a sleep-related metric such as, but not limited to, poor sleep quality, sleep disordered breathing burden, and/or disease burdens. In some examples, the stimulation therapy may comprise a neuromodulation therapy. The sleep treatment period could be a daily or nightly sleep treatment period and it is recognized that reference to one (e.g., nightly) could be substituted for the other (e.g., daily) throughout the examples provided.

In some examples, a patient having an IMD may be responsible for titrating (i.e. increasing or decreasing) a stimulation therapy amplitude over time. The present inventors have found that, often, the patient will increase their stimulation therapy amplitude too quickly and beyond a tolerable point (e.g. become uncomfortable, cause awakenings, etc.), which can lead to reduced stimulation therapy usage. In some cases, the patient may develop such a negative association with therapy that they may all together cease therapy. By evaluating patient therapy outcomes for multiple nightly sleep treatment periods and collecting data regarding patient therapy outcomes associated with differing stimulation therapy settings, the data can be plotted or otherwise evaluated to identify a solution stimulation therapy setting that maximizes usage while minimizing disease burden (i.e. any of a variety of adverse patient therapy outcomes). Once the solution stimulation therapy setting is identified, which may be at least partially determined via observing a later reduction in usage and/or detriment in therapy outcome(s), the current stimulation therapy setting can be adjusted/reduced to the solution therapy setting before patient outcomes are more negatively affected, which is believed to improve long-term chances of success with patient adherence and embrace of the device while achieving the desired therapeutic results. In the case where varying one stimulation therapy setting over a plurality of nightly treatment periods does not lead to the conclusion that one particular stimulation setting will increase usage while minimizing disease burden, a different stimulation therapy setting/parameter can be systematically varied (while keeping other stimulation therapy settings/parameters fixed). Therefore, various examples can be described as including tracking values of patient metrics over supra-day time period(s) (longer than one day (24 hour period)) and, based on the tracked values, incrementing or decrementing a value of the stimulation therapy settings.

At least some of the example devices and/or example methods may relate to sleep disordered breathing (SDB) care, which may comprise monitoring, diagnosis, and/or stimulation therapy. However, at least some examples of guiding stimulation therapy for a patient metric also may relate to cardiac care, drug delivery, pelvic-related care, and/or other forms of care, whether standing alone or in association with sleep disordered breathing (SDB) care.

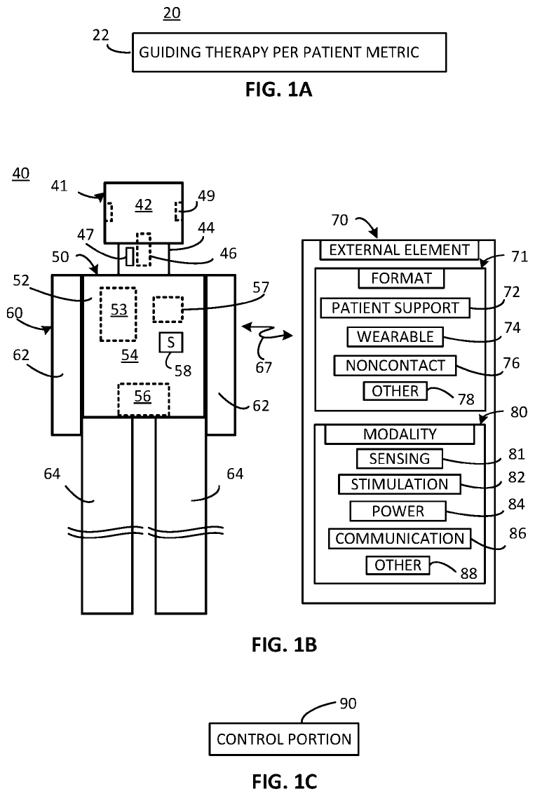

These examples, and additional examples, are further described in association with at least FIGS. 1A-9G.

As schematically represented at 20 in FIG. 1A, in some examples a method comprises guiding stimulation therapy for a patient metric at 22. In some examples, the stimulation therapy may comprise a neural therapy, and in some examples, the stimulation therapy may deliver simulation to a target nerve and/or muscle innervated by the target nerve. In some examples, this stimulation may target a junction of the nerve and muscle such as the terminal ends of the nerve fibers at/in the muscles which those nerves innervate.

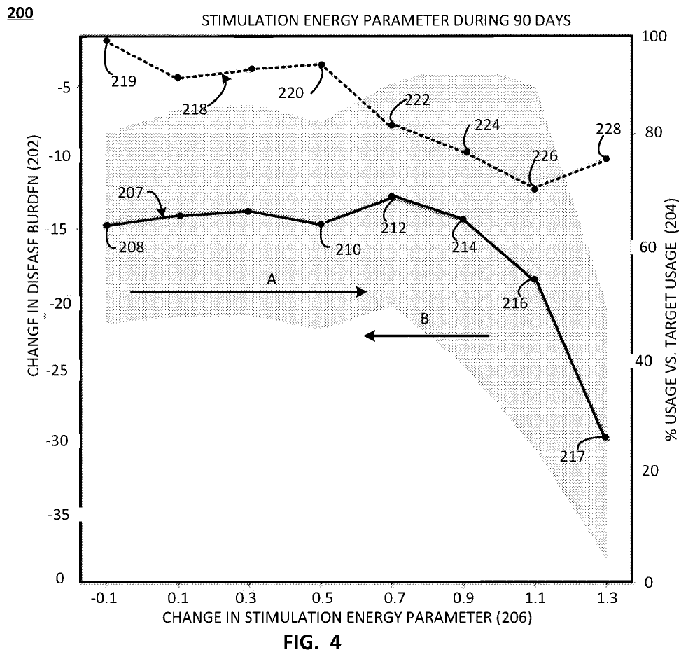

Example therapy outcomes can include any metric shown to improve health or a condition resulting from the stimulation therapy, any metric corresponding to the patient's use of, or adherence to, the stimulation therapy, and/or any other metric contributing to the well-being of the patient. In examples relating to sleep disordered breathing, the metric(s) may comprise, but are not limited to, an average apnea frequency per sleep treatment period (e.g. Apnea-Hypopnea Index-AHI); Epworth Sleepiness Scale (ESS); snoring; arousals; and patient perceived sleep quality. Examples are further described in association with at least therapy outcome (e.g., 113, 412) and patient metric, e.g. 112/114, 418, and 2916 in at least FIGS. 3, 6A and 9A, respectively.

In some examples, guiding the stimulation therapy may comprise guiding the stimulation therapy over a time period greater than a single stimulation treatment period (e.g., night). For instance, some example stimulation treatment periods may correspond to a daily or nightly treatment period, such as guiding the stimulation therapy with regard to a patient metric for a multiple number of daily or nightly treatment periods. Because the time window of guiding the stimulation therapy is longer than one day (24 hour period) in at least some examples, the time window may sometimes be referred to as a “supra-day” time period in some examples. In some examples, stimulation therapy is applied every night within the time period for the entirety or substantially the entirety of each nightly treatment period. Substantially the entirety may include, in some examples, brief pauses in stimulation therapy. In some examples, the brief pauses in stimulation therapy may be due to Wake After Sleep Onset (WASO). In some examples, stimulation treatment during a nightly treatment period is continuous in the sense that once started it is not intentionally interrupted for therapeutic goals. In one example, for each hour (or partial hour) that stimulation therapy is ON, therapeutic stimulation signals are delivered, according to parameters, whether that be open loop, closed loop, or various pulsed stimulation parameters. Once the solution setting is determined, the solution setting is applied 6 during subsequent nightly treatment periods, throughout each nightly treatment period, dependent on patient voluntary usage. In various examples, stimulation energy settings may be reduced to improve therapy outcomes but stimulation is not ceased during each respective nightly treatment period. As stated previously, in some examples, the nightly treatment periods can be substituted with daily treatment periods.

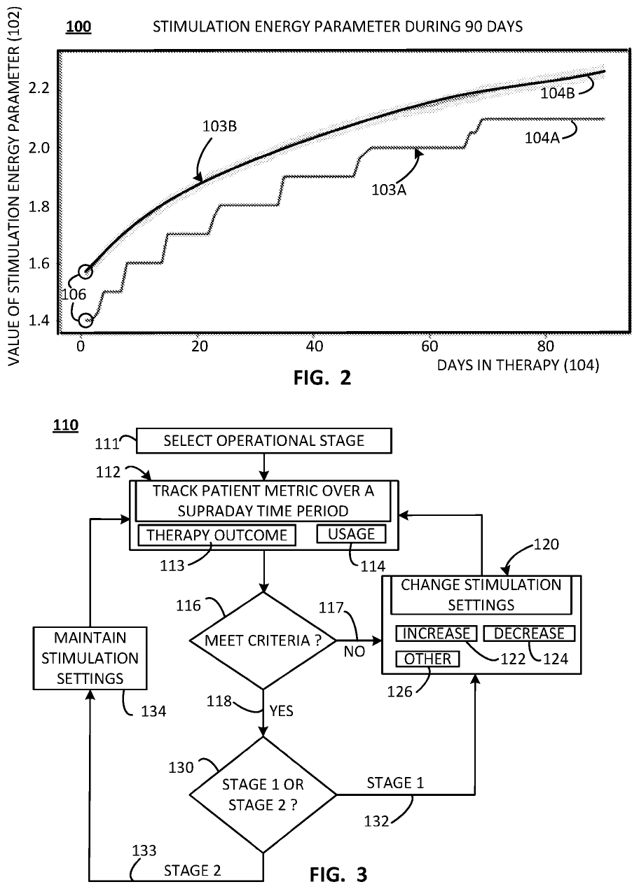

As previously mentioned and as further described below in association with at least FIGS. 2-9E, in some examples guiding stimulation therapy for a patient metric may be associated with or part of a method of providing stimulation therapy (e.g. neurostimulation) to patient tissue for improving a medical condition or disorder, such as, not limited to, sleep disordered breathing (SDB), pelvic disorders (e.g., incontinence), or other conditions amenable to peripheral nerve stimulation energy. However, examples are not so limited, and the example methods (and/or devices) for guiding therapy according to a patient metric throughout the present disclosure may be applicable to other forms of therapy.

By way of background, FIG. 1B is block diagram schematically representing a patient's body 40, including example target portions 41-64 at which at least some example sensing element(s) and/or stimulation elements may be employed to implement at least some examples of the present disclosure.

As shown in FIG. 1B, patient's body 40 comprises a head-and-neck portion 41, including head 42 and neck 44. Head 41 comprises cranial tissue, nerves, etc., and upper airway 46 (e.g., nerves, muscles, tissues), etc. As further shown in FIG. 1B, the patient's body 40 comprises a torso 50, which comprises various organs, muscles, nerves, other tissues, such as but not limited to those in pectoral region 52 (e.g., lungs 53, cardiac 57), abdomen 54, and/or pelvic region 56 (e.g., urinary/bladder, anal, reproductive, etc.). As further shown in FIG. 1B, the patient's body 40 comprises limbs 60, such as arms 62 and legs 64.

It will be understood that various sensing elements and/or stimulation elements as described throughout the various examples of the present disclosure may be deployed within the various regions of the patient's body 40 to sense and/or otherwise diagnose, monitor, treat various physiologic conditions such as, but not limited to those examples described below in association with FIGS. 1C-9E. In some such examples, a stimulation element 47 may be located in or near the upper airway 46 for treating sleep disordered breathing (and/or near other nerves/muscles for treating other conditions) and/or a sensing element 58 may be located anywhere within the neck 44 and/or torso 50 (or other body regions) to sense physiologic information for providing patient care (e.g. SDB, other) with the sensed physiologic information including, but not limited to, sleep onset detection and related parameters.

In some examples, at least a portion of the stimulation element 47 may comprise part of an implantable component/device, such as an implantable pulse generator (IPG) whether full sized or sized as a microstimulator. The implantable components (e.g. IPG, other) may comprise a stimulation/control circuit, a power supply (e.g. non-rechargeable, rechargeable), communication elements, and/or other components. In some examples, the stimulation element 47 also may comprise a stimulation electrode arrangement and/or stimulation lead connected to the implantable pulse generator.

Further details regarding a location, structure, operation, and/or use of the sensing element 58, external element(s) 150, and/or stimulation element 47 are described below in association with at least FIGS. 1C-9G.

In some examples, at least a portion of the stimulation element 47 may comprise part of an external component/device such as, but not limited to, the external component comprising a pulse generator (e.g. stimulation/control circuitry), power supply (e.g. rechargeable, non-rechargeable), and/other components. In some examples, a portion of the stimulation element 47 may be implantable and a portion of the stimulation element 47 may be external to the patient.

Accordingly, as further shown in FIG. 1B, the various sensing element(s) 58 and/or stimulation element(s) 47 implanted in the patient's body may be in wireless communication (e.g. connection 67) with at least one external element 70.

As further shown in FIG. 1B, in some examples, the external element(s) 70 may be implemented via a wide variety of formats such as, but not limited to, at least one of the formats 71 including a patient support 72 (e.g. bed, chair, sleep mat, other), wearable elements 74 (e.g. finger, wrist, head, neck, shirt), noncontact elements 76 (e.g. watch, camera, mobile device, other), and/or other elements 78.

As further shown in FIG. 1B, in some examples, the external element(s) 70 may comprise one or more different modalities 80 such as (but not limited to) a sensing portion 81, stimulation portion 82, power portion 84, communication portion 86, and/or other portion 88. The different portions 81, 82, 84, 86, 88 may be combined into a single physical structure (e.g. package, arrangement, assembly), may be implemented in multiple different physical structures, and/or with just some of the different portions 81, 82, 84, 86, 88 combined together in a single physical structure.

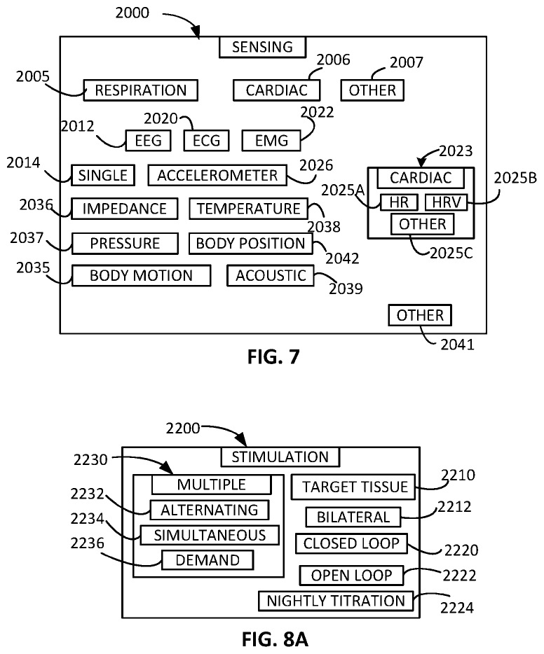

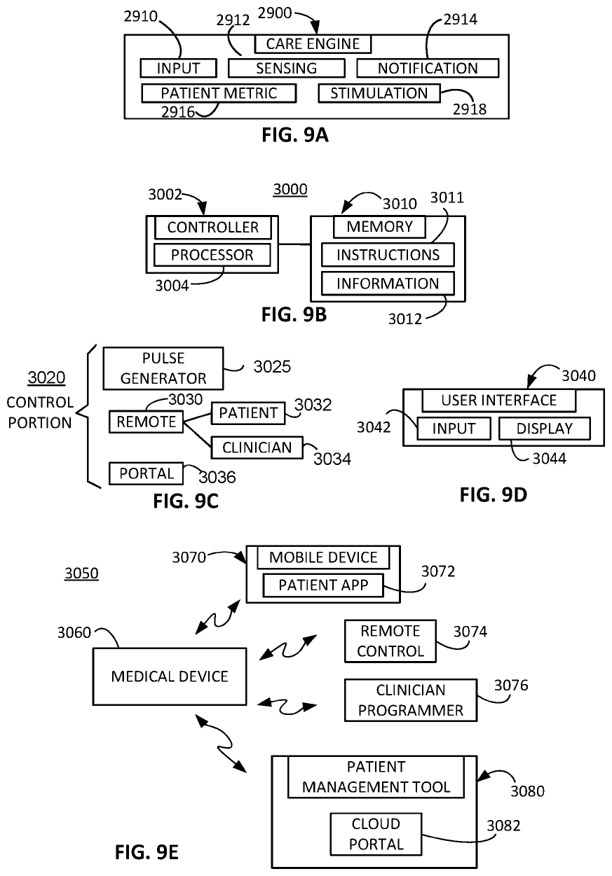

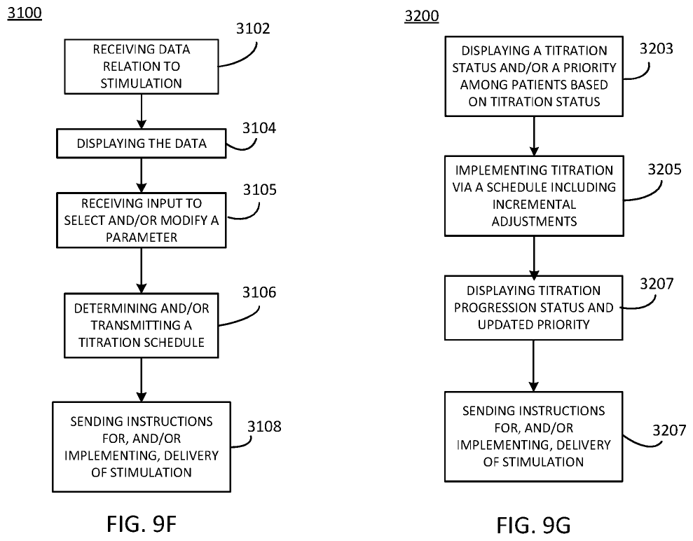

Among other such details, in some examples the external sensing portion 81 and/or implanted sensing element 58 may comprise at least some of substantially the same features and attributes of at least sensing portion 2000 and/or care engine 2900, as further described below in FIGS. 7 and 9A, respectively.

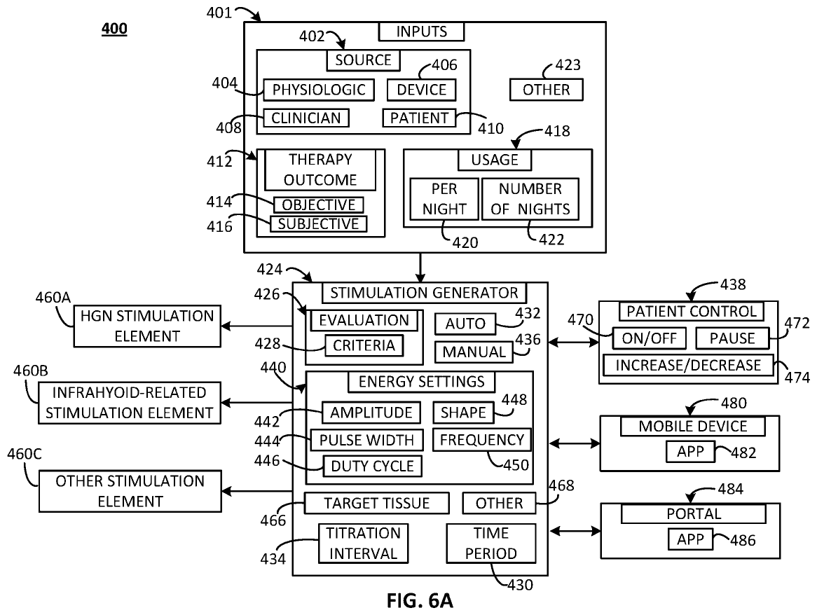

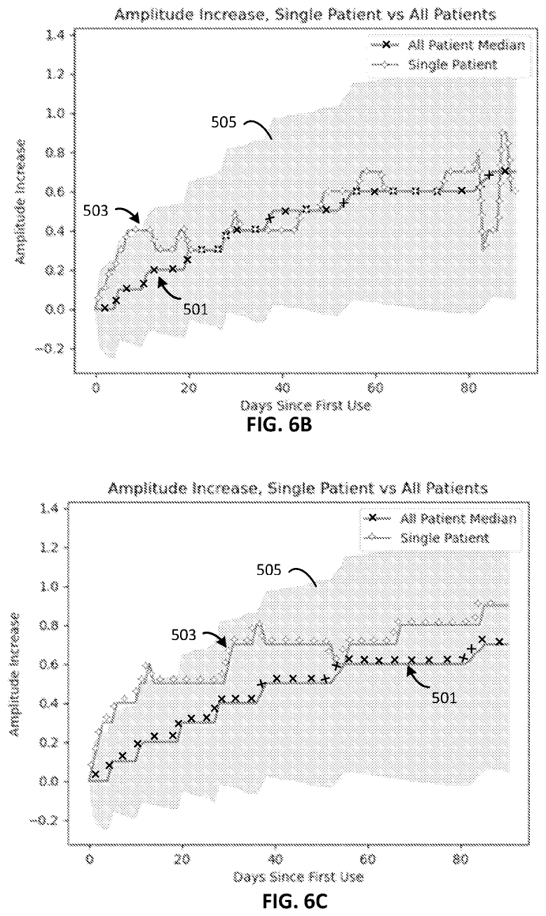

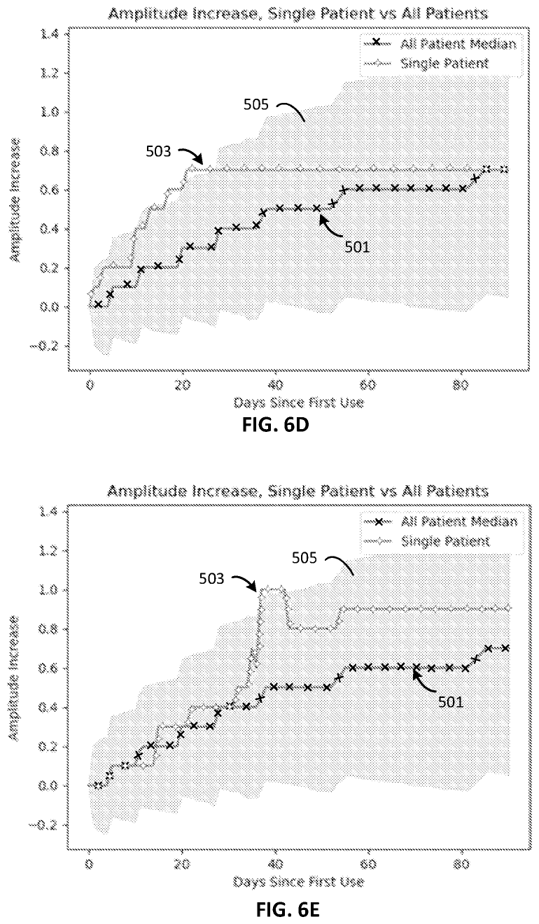

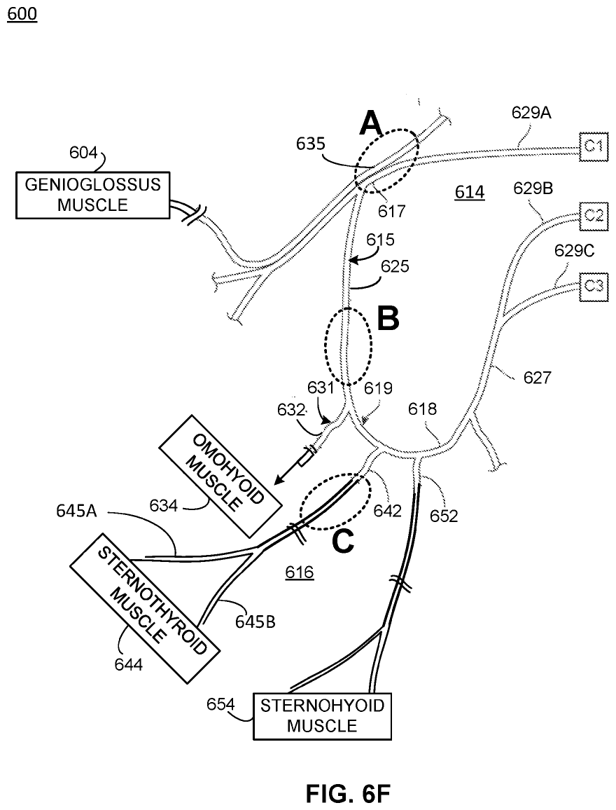

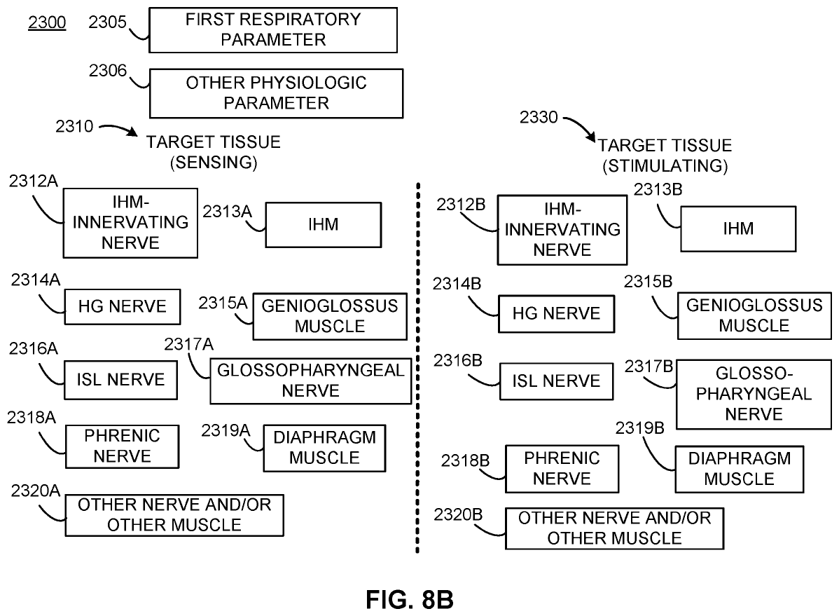

In some examples, the stimulation portion 82 and/or implanted stimulation element 47 may comprise at least some of substantially the same features and attributes of at least the stimulation arrangements, as further described below in association with at least FIGS. 6A and 8A and/or other examples throughout the present disclosure.

In some examples, the external power portion 84 and/or power components associated with implanted stimulation element 47 may comprise an example implementation of, and/or at least some of substantially the same features and attributes as, at least the stimulation arrangements, as further described below in association with at least FIGS. 6A, 8A and/or other examples throughout the present disclosure. In some such examples, the respective power portion, components, etc. may comprise a rechargeable power element (e.g. supply, battery, circuitry elements) and/or non-rechargeable power elements (e.g. battery). In some examples, the external power portion 84 may comprise a power source by which a power component of the implanted stimulation element 47 may be recharged.

In some examples, the wireless communication portion 86 (e.g. supporting and/or including connection/link at 67) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 88 via which other aspects of implementing medical care may be embodied in external element(s) 70 to relate to the various implanted and/or external components described above.

FIG. 1C schematically represents a control portion 90, which may comprise at least some of substantially the same features and attributes as the control portion 3000 of FIG. 9B and/or care engine 2900 in FIG. 9A. In some examples, the control portion 90 will be part of a care engine (e.g., 2900) or the like. Among other aspects, example methods and/or example devices may be implemented via the control portion 90. In some examples, the control portion 90 may be used to implement at least some of the various example devices and/or example methods of the present disclosure as described herein. In some examples, the control portion 90 may form part of, and/or be in communication with, the stimulation element (e.g., 47 in FIG. 1B), sensing element 58, and/or other medical device (e.g., pulse generator or the like).

In various examples, the control portion 90 is programmed to guide stimulation therapy for a patient metric. In one example, the control portion 90 is programmed to guide over a supra-day time window, the supra-day time window comprising at least 7 or at least 14 or at least 30 or at least 60 or at least 90 days. In one example, the control portion 90 is configured to track, from a first patient therapy point of electrical stimulation of at least one upper airway patency-related tissue by a stimulation element, changes in stimulation energy values for the patient over a multiple number of sleep treatment periods toward a solution stimulation energy parameter value at which criteria are met for: 1) a target therapy outcome parameter; and 2) a target usage parameter. In one example, the solution stimulation energy parameter value is an amplitude value. The first patient therapy point may, in some examples, be an initial patient therapy point that is the first nightly treatment period for the patient. These and additional aspects of the control portion 90 and its configuration will be further discussed below. In some instances, the values of stimulation energy parameters may sometimes be referred to as stimulation energy settings, and/or the values of stimulation energy parameters at a solution (for target therapy outcome and/or usage) may sometimes be referred to as solution stimulation settings.

As shown in FIG. 2, in one example, methods of the disclosure include tracking (e.g. via graph 100), values in a stimulation energy parameter 102 (e.g., amplitude; y-axis) over the course of a first number of days (i.e. time window) in a treatment period 104 (x-axis). It will be understood that graph 100 represents just one example. Among other uses, in some examples such tracking may be used as part of example methods including determining (e.g. titrating) and guiding therapy according to a patient metric such as, but not limited to, guiding values of stimulation therapy parameters based on disease burden (e.g. therapy outcome) and/or patient usage of the therapeutic device.

While the graph 100 in FIG. 2 depicts increases of values of an amplitude setting over a time-window, in some examples the values of another, different parameter (e.g. pulse width, frequency, etc.) of stimulation energy parameter may be changed over time until a target value of the particular stimulation energy parameter is achieved. In some examples, values of one stimulation energy parameter may be varied while the value of other stimulation energy parameters may remain fixed, as further described later in association with at least FIGS. 6A, 8A, and 8B.

As shown in FIG. 2, line 103A depicts stepwise increases in the values of a stimulation energy parameter 102 (e.g. amplitude values), with more frequent increases being made earlier (e.g. first few days, weeks) in the multi-day time window (e.g., 90 days) and less frequent changes being made later (e.g. last month, weeks) in the multi-day time window (e.g. 90 days). In some such examples, line 103A may represent at least some of the most common values of a stimulation energy parameter (e.g. amplitude) during an initial therapy period such as, but not limited to, a first 90 days of therapy.

In some examples, each stepwise increase represented by line 103A is made by generally the same value such as, but not limited to, 0.1 Volts in some examples. However, in some examples, the interval between changes may increase as the value grows during the time window and the value of the stimulation energy parameter 102 (e.g. amplitude) may be approaching or have already reached a target value when the end of the time-window is reached.

Meanwhile, line 103B in FIG. 2 also depicts increases in the values of the stimulation energy parameter 102 (e.g. amplitude), except expressed as a smoothed trajectory (e.g. path) of the changing values over the supra-day time window 104. In some such examples, the target value of the stimulation energy parameter 102 may be the same as, or less than, a device maximum limit (e.g. set by manufacturer) and/or a particular patient's therapeutic maximum limit (e.g. set by clinician).

FIG. 3 schematically represents an example method and/or device 110 for guiding stimulation therapy according to a patient metric. In some examples, the example method and/or device may comprise an example implementation of, and/or at least some of substantially the same features and attributes as, the examples of FIGS. 1A-2. Accordingly, as shown in FIG. 3, in some examples the method (and/or device) 110 may comprise selecting operational stages 111 by which the stimulation therapy may be guided. In some such examples, guiding stimulation therapy may sometimes include an initial stage (e.g. Stage 1) and one or more later steady-state stages (e.g. Stage 2) with both stages being used to manage the values of stimulation energy parameters toward meeting a patient metric.

As further shown in FIG. 3, at 112 the example method 110 further includes tracking (which can include recording) at least one patient metric (e.g., therapy outcome 112 and/or usage 114) over a time period (e.g., a plurality of days, see also time period 104 in FIG. 2), and evaluating the at least one patient metric 112, 114 as the stimulation therapy energy changes (102 in FIG. 2) over the time window (104 in FIG. 2) in comparison to a criteria at 116. In one example, the criteria at 116 may comprise a particular value of target therapy outcome 113 and a particular value of a target usage 114. If the criteria is not met at 116 (e.g. NO at 117), the stimulation settings may be changed at 120. As shown at 120, values of a particular stimulation energy parameter may be increased 122, decreased 124, or other changes 126 in the stimulation settings may be made, such as changing other stimulation energy parameters, as further described later in association with at least FIG. 6A.

As just one example, the settings (e.g. values) of stimulation energy are increased 122 via one or more parameters in attempts to further improve the therapy outcome(s) 112, while maintaining or increasing usage 114. With this in mind, it is believed that generally, for most patients, in the initial stage (e.g. initial titration or Stage 1), as values of a stimulation energy parameter (e.g. therapy amplitude) increase, therapy outcomes will correspondingly improve (e.g. disease burden will be lessened), until a point is reached at which further increases in the values of the particular stimulation energy parameter will cause usage to be detrimentally affected and/or cause therapy outcomes to decline instead of improving.

Accordingly, after changing the stimulation settings at 120 such as an increase, the method includes further tracking, which can include recording/storing (see also memory 3010 of FIG. 9B), the patient metric(s) at 112 over some portion (e.g. days, weeks) of the supra-day time window to determine if the criteria (at 116) are met. If the criteria are still not met (e.g. NO), then path 117 is taken again to further change the stimulation settings at 120, such as an increase to attempt further improvements in the patient metric(s) as observed via tracking at 112, and further evaluation at 116. This loop (e.g. along path 117, including 120, 112, 116) is repeated until a target patient metric is reached when the criteria (at 116) are met (e.g. YES) at which time path 118 is taken for determination of future actions in the example method of guiding therapy.

FIG. 2 illustrates one such example graphically representing at least a portion of an initial stage (e.g. Stage 1) of guiding therapy in which a progressive increase in values of an stimulation therapy energy parameter 102 such as amplitude (e.g., from about 1.4 to about 2.2 Volts) is tracked over a supra-day time period 104. In some examples, the supra-day time period may correspond to an initial therapy period which occurs after the patient is fitted with (e.g. ready to use) the therapeutic medical device, whether its components are implantable or external, or a combination of implantable components and external components. For a medical device including at least some implantable components, the supra-day time period may correspond to an initial therapy period which occurs post-implant once the implantable device/components have sufficiently stabilized within the patient's body and in which an initial stage of guiding (e.g. titrating) the stimulation therapy occurs.

In the example of FIG. 2, the supra-day time period is 90 days, however, the supra-day time period can include more or fewer days (i.e. may be 7 or more days, 14 or more days, 30 or more days, 60 or more days, 90 or more or fewer days).

With further reference to at least FIG. 2, in some examples, the initial stage (e.g. Stage 1) may include a plurality of nightly treatment periods including a first nightly treatment period (106; FIG. 2) through a first future nightly treatment period in both which usage and therapy outcome(s) have improved over time (as the stimulation energy has increased). Accordingly, Stage 1 may generally, in some examples, be considered a ramping up stage as evident from the generally ramped shape of lines 103A, 103B in FIG. 2.

Referring again to the example method (and/or device) 110 of guiding therapy in FIG. 3, after multiple successive increases in the value of the stimulation energy (e.g. along path 117) occur until the criteria regarding the patient metric are met at 116 (e.g. YES), the method proceeds along path 118 to 130 at which different paths (e.g. 132 or 133) may be taken depending on a stage of guiding therapy.

In some examples, path 132 may be provided for the initial stage (“Stage 1”) of guiding therapy. During Stage 1, it is possible that therapy outcomes and usage will meet the criteria at 116, however, via paths 118, 132 the example method may further increase (122) the stimulation settings at 120 (beyond the point of criteria being met) to identify if even better therapy outcomes and usage are obtainable.

Accordingly, a loop may be repeated along paths 118, 132 (including 120, 112, 116, 118, 130) for one or more iterations to determine if continued increases (122 at 120) in the values of stimulation energy parameter(s) result in continued improvement in the patient metric (at 112, 116).

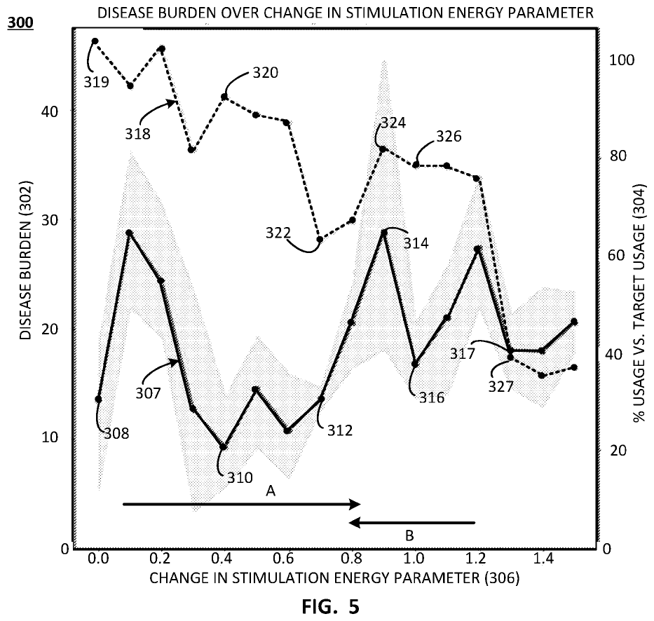

However, at some point, further increases (122 at 120) in the value of stimulation energy parameters will result in the patient metric (e.g. usage 114 and/or therapy outcome 113) no longer being met at 116 (e.g. NO at 117), such as the usage declining and/or therapy outcome declining despite the recent further increases in stimulation energy. Once this point is reached, the method of guiding therapy again takes path 117 to 120 to change stimulation settings via a decrease (124) the value of the stimulation energy parameter(s) in an attempt to re-direct the stimulation therapy to a solution at which the criteria (116) are met for the patient metric. For examples, one of the previous settings (e.g. value) of the stimulation therapy energy parameter may be identified just prior to a decline of the therapy outcome and/or usage with such previous settings being designated as a solution stimulation setting in this example. Accordingly, at 120, via the increase, decrease, and/or other parameters 122, 124, 126, the stimulation settings are returned to values at which the therapy outcome 113 and usage 114 were at or near their peak prior to seeing their decline upon further increases in stimulation energy. Demonstration of at least these aspects of the example method of guiding therapy are further illustrated, at least in part, in the later discussion of at least FIGS. 4 and 5.

Time periods occurring after completion of Stage 1 (e.g. such as, but not limited to, identification of the first future nightly treatment period), would be categorized under “Stage 2” 133 in the flow chart of FIG. 3.

In various examples, if the criteria is met at 116 and at 130 it is confirmed that the patient is in Stage 2, path 133 is taken by which the stimulation settings are maintained at 134 because the stimulation settings (e.g. value of the stimulation energy parameter(s)) are generally achieving the desired therapeutic outcome 112 and maintaining usage 114 as prescribed by the criteria at 116. The method continues to track the patient metric at 112, and at selectable period intervals, it is determined if the criteria (at 116) are met. As long as the answer is YES, then the method continues guiding therapy via path 133 (including 134, 112, 116, 118, 130, 133) to maintain delivering therapy at a patient metric (e.g. 113, 114) which meets the criteria at 116.

However, at a certain point in Stage 2, the criteria may no longer be met (e.g. NO) at 116, such that the flow of guiding therapy changes to take path 117 so that the stimulation settings are changed at 120, which may include an increase in the stimulation setting 122, a decrease in the stimulation setting 124 or some other change in the stimulation settings 126. In one example, if an AHI or snoring (e.g. 113) is increasing, for example, the change energy setting at 120 may be an increase in the stimulation energy parameter (e.g. amplitude). In one example, if the therapy outcome parameter 112 comprises an indication of an increase in arousals (e.g. 113) then the criteria at 116 would no longer be met, with the method then taking path 117 to permit the stimulation setting to be decreased via parameter 124 at 120 and further tracked at 112 to determine if the changed value of the stimulation energy parameter resulted in the therapy outcome being met at 116.

In some examples, other patient metrics and associated criteria may be used in addition to, or instead of, therapy outcome and usage to determine stimulation solution settings. In some such examples, at least some of these other patient metrics may relate to effects on a patient if or when the stimulation energy starts to become disruptive to sleep such as, but not limited to, relative comfort during stimulation.

In some examples in Stage 2 in which the criteria are being met at 116, and the method of guiding therapy maintains current values of stimulation settings (at 134), the example method may selectively and/or periodically take path 132 from 130 as if it were Stage 1 to change the stimulation settings to determine if a decrease in stimulation energy may still result in meeting the criteria. Some examples which might result in the patient needing a lower stimulation energy setting might include the patient losing weight, other health changes, surgical interventions, and the like. Similarly, in some examples, assuming on-going operation in Stage 2, the example method may selectively and/or periodically take path 132 from 130 as if it were Stage 1 to change the stimulation settings to determine if an increase in stimulation energy may still result in meeting the criteria. In this way, the example method (and/or example device) can periodically check to see if the patient metric (e.g. therapy outcome, usage) may be improved.

For both Stages 1 and 2, patient metrics are continued to be tracked at 112 over supra-day time periods for comparison to criteria at 116 for potential stimulation settings change at 120 according to examples of the disclosure.

The criteria for application at 116 can be implemented in many different ways according to examples of the disclosure. In one example, the criteria may include a threshold. In some examples, the criteria is met when the patient metric is at or above the threshold and, in other examples, the criteria is met when the tracked patient metric is at or below the threshold. In some examples, the criteria at 116 may be implemented as a percentage or relationship instead of, and/or in addition to implementation via a threshold.

Moreover, at selectable periodic intervals, the criteria may be adjusted to accommodate changes in patient health, conditions, needs, etc. and/or upon changes in other aspects of stimulation therapy, such as stimulation additional/different target tissues.

It is envisioned that, in some examples, criteria may include weighting at least some patient metrics or otherwise prioritizing patient metrics to prioritize one metric over the other. In some such examples, such weighting can be, in some examples, patient specific and dependent on particular concerns or objectives for the particular patient. For example, perhaps maximizing usage may provide more perceived value for one patient whereas maximizing an AHI reduction (e.g. greater reduction in disease burden) may have a greater perceived value for another patient. For instance, in one non-limiting example a patient may indicate that they want to stop snoring so their partner can sleep, whether or not meeting this objective would provide the best overall therapy outcome regarding other aspects such as AHI. For this patient, the criteria may be adjusted to expand a target range of usage hours operating under an assumption (which may be evaluated) that less usage at higher stimulation energy settings may result in less overall snoring.

In some examples, guiding therapy according to example of method 110 at least FIG. 3 may be performed at least partially automatically (432) and/or manually (436), as further described later in association with at least FIG. 6A.

Referring now to FIG. 4, which stands in addition to the previously described examples, FIG. 4 graphically illustrates aspects of the example of at least some aspects of the method of FIG. 3 for a first patient. In this example a graph 200 illustrates, via line 207, a first patient's changes in Disease Burden 202 along the left Y-axis (which may comprise AHI in some examples as one aspect of therapy outcome) and via line 218, a percentage of actual usage of the stimulation therapy 204 (i.e. when the stimulation therapy is ON) when the patient is asleep relative to a target usage (right y-axis) as values of a stimulation energy parameter (e.g. amplitude) is varied as represented along the x-axis 206 (“Change in Stimulation Energy Parameter). It can be seen at point 208 along line 207, for a given value of stimulation energy parameter (e.g. amplitude), the usage 219 (along line 218) is high and an approximately −15 change in AHI is observed (at 208) relative to a baseline disease burden that was present prior to delivery of stimulation therapy. In some examples, the absolute value of stimulation energy parameter (e.g. amplitude) at point 208 may generally correspond to a value of stimulation energy parameter (e.g. amplitude) such as or near points 104A or 104B in FIG. 2 in which most or all of the increases in values of the stimulation energy parameter has already occurred in a ramped manner over an initial titration period (e.g. Stage 1) of the time window 104.

With further reference to FIG. 4, in this example method, a value of stimulation energy parameter (e.g. amplitude) is increased (Direction A) from point 208, which may correspond to latter portion of a Stage 1, ramp up stage, as described above in relation to FIGS. 2-3. At point 210 along line 207 in FIG. 4, a cumulative increase in the value of the stimulation energy parameter (e.g. amplitude) from point 208 of 0.5 has occurred and usage remains high at 220 along line 218, while there is still approximately an about −15 change in disease burden (e.g. AHI). For this example patient, via the method of guiding therapy, at point 212 (along line 207) stimulation amplitude is again increased (Direction A) to a 0.7 change at which usage 222 (along line 218) decreases and the change in disease burden drops (i.e. a lesser reduction in AHI; worse therapy outcome). In one example, this reduction in usage and reduction in disease burden (e.g. AHI) change could identify a solution stimulation setting being that of the previous setting, i.e. the stimulation setting at point 210. In one example, once the solution stimulation setting is discovered, the solution stimulation setting may be implemented for further nightly treatment periods.

In some examples, the solution stimulation setting may not be considered discovered until a trend in decreased usage is identified. In one example, this trend may be a decrease in usage lasting two or more nights. In the example of FIG. 4, such a trend can be seen at points 212/222, 214/224, 216/226 and 217/228.

Referring now in addition to FIG. 5, which graphically illustrates aspects of another example of the method of FIG. 3 for a second patient. In some examples, this example method and/or device may comprise an example implementation of, and/or at least some of substantially the same features and attributes as, the examples of FIGS. 1A-4.

This example provides a graph 300 illustrating the second patient's disease burden 302 (e.g. AHI as an example therapy outcome) plotted as line 307 and, plotted as line 318, a percentage of usage of the stimulation therapy 304 (i.e. when the stimulation therapy is ON) when the patient is asleep as compared to a target usage as a value of stimulation energy parameter (e.g. amplitude) 306 is varied (e.g. changes in amplitude) over some time period. For the second patient of FIG. 5, an 19 increase in amplitude (in Direction A) as represented from point 308 to point 310 to point 312 to point 314 to point 316 to point 317 of line 307 did not lead to better AHI 302, as revealed by the general trend of increases in AHI from points 312 to 327. During this same time period and changes in amplitude, the second patient's usage at points 320, 322, 324, 326, 327 (from initial point 319) decreased as amplitude increased. Although the decrease in usage from points 319 to point 327 was not continually negative, usage at point 318 is significantly higher than those to the right of point 320 along line 318. At point 320 along line 318 representing usage, the AHI is correspondingly low at point 310. Therefore, in one example, one stimulation solution setting may be identified/discovered at point 310 along line 307 and 320 along line 318. Point 310/320 is one in which, at that change in amplitude setting 306, the AHI 302 is relatively low, while maintaining relatively high usage 304. In one example, once the solution stimulation setting is discovered as illustrated via FIG. 5, a Stage 1 operation of the example method 110 of guiding therapy in FIG. 3 may be considered complete.

For either of the examples of FIGS. 4 and 5, once a solution stimulation setting is discovered (e.g. Stage 1), various example methods can further include continuing to guide the patient metric over a supra-day time period under a Stage 2 operation as described above with respect to FIG. 3. In one example aspect of Stage 2 (FIG. 3), the patient metric is tracked over a supra-day time period, which can include a therapy outcome and usage similar to the Stage 1 analysis. If the metrics meet criteria, stimulation settings are maintained (e.g. 134 in FIG. 3). If the metrics do not meet the criteria (e.g. path 117 in FIG. 3), stimulation settings are changed (e.g. 120 in FIG. 3), which could mean an increase (122) in stimulation settings, decrease (124) in stimulation settings or other change (126) believed to improve the desired patient metrics so that future tracked patient metrics are more likely to meet the criteria.

It is to be understood that the use of AHI as an indicator of therapy outcome of the examples of FIGS. 4 and 5 is merely an example of many different types of therapy outcomes for various types of disease burdens and that other disclosed therapy outcome/patient metrics (e.g. Epworth Sleepiness Scale (ESS), etc.) of the disclosure can be similarly evaluated in accordance with the disclosure alone and/or in combination with AHI.

Referring now in addition to FIG. 6A, which illustrates various components of the devices and methods of the disclosure in additional detail. FIG. 6A schematically represents an example device 400. In some examples, the example device 400 of FIG. 6A may comprise an example implementation of, and/or at least some of substantially the same features and attributes as, the examples described in association with FIGS. 1A-5.

In general terms, example device 400 provides for guiding stimulation therapy according to a patient metric. Among other aspects, example device 400 generates stimulation settings via generator 424 by which stimulation may be delivered to a target tissue via stimulation elements (e.g. 460A, 460B) and may do so based on receiving information from input portion 401. Moreover, the stimulation generator 424 (and other components of device 400 such as (but not limited to) devices providing inputs via input portion 401) as a whole may be in wireless communication with a patient control 438 and/or app on a mobile device 480 or portal 484 with such communication regarding control signals, data (including therapy information/settings/results), power, and/or other parameters, etc. At least some aspects of such communication is further described later in association with at least FIGS. 9A-9E.

With this in mind, example methods and devices of the disclosure comprise a variety of inputs made available via input portion 401. Such inputs can include, for example source-derived inputs 402 such as physiologic inputs 404, device inputs 406 (e.g., from device 3060 discussed below), clinician inputs 408 and patient inputs 410. At least some physiologic inputs are further described later in association with at least FIG. 7.

Inputs from input portion 401 can further include therapy outcomes portion 412, whether they be objective 414 or subjective 416 therapy outcomes, for example. In one example, one or more inputs 401 can include usage inputs 418 either for one individual nightly treatment period 420 (e.g. hours per night) and/or for a plurality of nightly treatment periods 422 (see also tracked usage 204, 304 as shown in FIGS. 4 and 5, for example).

The present disclosure is not intended to be limited to these specific inputs and other inputs 423 may be utilized in examples of the disclosure.

In various examples, one or more inputs 401 are sent to and received by the stimulation generator 424 of the medical device (e.g. implantable), such as the medical device 3060 described below with respect to FIG. 9E. In one example, the stimulation generator 424 is configured to conduct one or more of the processes, operations, etc. of FIG. 3. In some examples, the stimulation generator 424 may comprise at least a partial implementation of, and/or comprise at least some of substantially the same features and attributes as, the control portion of FIGS. 1C, 9A-9E. Therefore, in some examples, the stimulation generator 424 includes an evaluation portion 426 is configured to evaluate one or more inputs 402 from input portion 401 in comparison to criteria 428.

Via an energy settings portion 440, the example stimulation generator 424 may track, control, generate, and/or direct generation of stimulation signals, according to stimulation energy settings, to be delivered via one or more stimulation elements (e.g., stimulation elements 460A, 460B). Such individually controllable settings which may be selected, adjusted, etc. may comprise one or more of stimulation amplitude 442, stimulation pulse width 444, stimulation duty cycle 446, stimulation shape 448, and stimulation frequency 450, for example.

In one example, the stimulation generator 424 may facilitate control over stimulation therapy energy via energy settings portion 440 over a selectable plurality of days as designated via time period parameter 430. For instance, in one example, the time period 430 comprises a supra-day time period (i.e. greater than one day (24 hour period)). In some examples, the time period may be at least 7 days, at least 14 days, at least 30 days, at least 60 days, or at least 90 days, such as previously described in association with at least FIGS. 2-5.

In some examples, such control may be exerted by stimulation generator 424 to deliver changes in stimulation therapy energy over a supra-day period corresponding to a ramping up or Stage 1 phase, as previously described in association with at least FIGS. 3-5, with such changes comprising increases or decreases as prudent to achieve a stimulation solution setting for the stimulation energy parameters in accordance with a patient metric.

In one example, the stimulation generator 424 may be configured to, and be selectable to, operate in an automatic mode 432 in which the stimulation energy settings 440 for a stimulation signal are automatically titrated to determine a stimulation solution setting for stimulation energy parameters based on a protocol such as, but not limited to, at least some of substantially the same features and attributes of the arrangements of at least FIGS. 2-5. In some such examples, a stimulation energy adjustment or change may be made independently without the patient's action or input. In the example where the stimulation setting is automatically titrated (automatic mode 432), the titration interval 434 may be controlled by the stimulation generator 424. In some examples, the titration interval 434 may be some selectable number of days, weeks, months, and at the selected interval, the stimulation generator 424 may use inputs from input portion 401, such as therapy outcome, usage, and/or other information to evaluate the effectiveness of the current stimulation energy settings 440 and determine if a change to such settings should be made, whether in accordance with the example arrangements of FIGS. 2-5 and/or arrangements other than those described in FIGS. 2-5.

In some examples, the stimulation generator 424 may comprise, and operate under, a manual mode 436 in which a patient is instructed, via a clinician or otherwise via a device (e.g. app), to manage the stimulation energy settings (i.e. increase or decrease the stimulation setting) via a patient control 438. For example, the stimulation generator 424 may be configured to assist the patient in this task via receiving one or more inputs 401, such as therapy outcome 412 and usage 418 (for evaluating in comparison to criteria 428) and communicating this information to the patient. In some examples, this arrangement may further comprise communicating to the patient suggested changes to a value of a stimulation energy parameter in order to implement a route for likely arrival at a stimulation solution setting, as previously described in association with at least FIGS. 2-5 and FIG. 3 in particular. In some examples, in a first stage (Stage 1) of determining a solution stimulation setting to produce a therapy outcome (113 in FIG. 3; 412 in FIG. 6A) and/or usage (114 in FIG. 3; 418 in FIG. 6A), which meets a criteria (116 in FIG. 3; 428 in FIG. 6A), the stimulation generator 424 (including or in communication with a control portion) may first just observe a patient applying manual control (per mode 436) of values of a stimulation energy parameter (e.g. amplitude) as generally instructed by a clinician without the stimulation generator guiding or directing such patient choices. During such observation, the stimulation generator 424 may observe the patterns illustrated in FIGS. 2, 4-5, and then identify a stimulation solution setting for amplitude (and/or other stimulation energy parameters) which may then be communicated to the patient and/or to the clinician for use in on-going stimulation therapy (e.g. Stage 2 operation in FIG. 3).

With further reference to FIG. 6A, the stimulation generator 424 may also control signals to designate the target tissue (nerve and/or muscle) 466 in some examples. For example, the stimulation generator 424 may be in communication with one or more stimulation elements 460A, 460B, 460C. In one example, at least one stimulation element is a hypoglossal nerve stimulation element 460A. In one example, at least one stimulation element is an infrahyoid-related nerve stimulation element 460B. In one example, at least one stimulation element is an other stimulation element 460C, which can be any desired target tissue (nerve and/or muscle). In some examples, at least two of a hypoglossal nerve stimulation element 460A, an infrahyoid-related stimulation element 460B, and an other stimulation element 460C are provided as it may be determined, via methods of the disclosure or otherwise, that stimulation of one nerve is more effective or otherwise advantageous than the others. In yet another example, it may be determined that stimulation of two or more nerves and/or muscles is most effective or otherwise advantageous. In various examples, the stimulation elements 460A, 460B, 460C receive control or stimulation signals from the stimulation generator 424 to implement stimulation therapy to the desired target tissue. In some examples, each target tissue may have its own/different set of stimulation energy settings because of a different location, behavior, and/or effect of the muscles (innervated by the respective different target nerves) on the target anatomy (e.g. upper airway patency) and/or because of the manner in which the particular nerve is accessed, whether stimulation is applied at more distal or proximal locations along the nerve, etc. Moreover, the act of stimulating of a combination of different target nerves also may affect the most efficacious stimulation energy parameters (settings) for selection for each respective target nerve so that the multiple target nerve stimulation may be implemented in a complementary manner. Further details regarding applying stimulation to one or more target tissues is described later in association with at least FIGS. 8A and 8B.

The stimulation generator 424 may have other 468 functions and capabilities in alternate examples to facilitate the methods and processes of examples of the disclosure.

In some examples, at least a portion of the stimulation generator 424 may comprise part of an implantable component/device, such an implantable pulse generator (IPG) whether full sized or sized as a microstimulator. However, in some examples, at least a portion of the stimulation generator 424 may comprise part of an external component/device. In some examples, a portion of the stimulation generator 424 may be implantable and a portion of the stimulation generator 424 may be external to the patient.

In some examples, examples methods may utilize a patient control 438, which can be a patient remote or the like, for various purposes such as communication of control signals, data, power, etc. to and from the stimulation generator 424, among other components. One or more purposes of the patient control 438 may include turning stimulation therapy on or off using the on/off function 470 to begin or terminate a therapy session. In some examples in which automatic initiation, automatic titration, etc. of stimulation therapy is implemented via stimulation generator 424, the on/off function 470 may provide a manual override function to initiate or terminate therapy. Additionally, in some examples, the patient control 438 may comprise a pause function 472 to momentarily suspend stimulation therapy but without terminating a therapy session during a nightly treatment period. In one example, the patient control 438 may be configured to allow the patient to change their stimulation energy setting 474 (i.e. increase or decrease) in the manual mode 436 described above, for example, which may include normal operation (e.g. Stage 2 in FIG. 3) or initial operation (e.g. Stage 1 in FIG. 3).

In some examples, the patient may be prompted by their clinician on which values of stimulation energy parameters (e.g. amplitude) may be most effective for their situation. In various examples, the patient may be guided by their clinician based on the patient's own data and/or based on data derived from other patients. Such guidance may occur at least via a clinician: (1) programming the stimulation generator 424 via nearby wireless communication (e.g. in the office); (2) programming the patient control 438 (or an app 482 on a patient mobile device 480) to limit a range of increases or decreases of stimulation energy parameters (e.g. amplitude); (3) programming the stimulation generator 424 via an app 486 of a portal 484 (e.g. web or cloud resource), as further described below regarding FIG. 6A and FIGS. 9A-9G; and/or (4) other means.

In some examples, the patient control 438 may be implemented via at least some of substantially the same features and attributes as remote control 3074 of FIG. 9E and/or remote 3030,3032 of FIG. 9C in association with FIGS. 9A-9E.

Various examples may include a mobile device 480 comprising an application 482 configured to provide one or more inputs from input portion 401 (e.g., patient input 410) and/or provide any of the functions of patient control 438. In some examples, the mobile device 480 and application 482 may be utilized to communicate with the patient regarding their therapy. In some examples, the communication may be regarding a notification, which may prompt the patient to adjust their stimulation settings for future nightly treatment periods. In one example, the application 482 may be configured to prompt the patient to confirm receipt or implementation of the communication/instructions via the application. It will be understood that a mobile device 480 can be used in combination with or as a substitute for the patient control 438 in some examples. In some examples, the mobile device 480 (and/or app 482) may be implemented via at least some of substantially the same features and attributes as mobile device 3070 (and/or app 3072) of FIG. 9E in association with FIGS. 9A-9E.

Some examples may include a clinician portal 484 that may comprise an application 486 configured to provide one or more clinician inputs 408, which can include, but are not limited to prescribed criteria, stimulation energy settings (in 440), a value (e.g. duration) of supra-day period 430, and a value (e.g. duration) of the titration interval 434. It will be understood that, in some examples, prior to beginning an example method of initial titration (e.g. Stage 1 in FIG. 3) and/or later steady-state titration (e.g. Stage 2 in FIG. 3), a clinician would have already programmed the stimulation generator 424 (and/or input portion 401, other components) to select settings (e.g. values) for the stimulation energy parameters (e.g. amplitude, pulse width, shape, pulse width, frequency, duty cycle) of the stimulation signal to be delivered to the patient, depending on the target tissues, patient size/condition, etc. For most or all patients, once these stimulation energy parameters are set by the clinician, they cannot be changed by the patient except for a limited range of changes which can be made to the amplitude setting.

However, it will be noted that in examples in which the initial stage (e.g. Stage 1 in FIG. 3) of titrating the value of stimulation energy parameters is performed automatically without patient control, some of the other stimulation energy parameters (e.g. pulse width 444, frequency 450, shape 448, duty cycle 446) also may be varied in addition to, or instead of, varying the value of the amplitude parameter in an effort to identify a stimulation solution setting for the group of stimulation energy parameters, such as via the example of FIGS. 2-5.

In some examples, the clinician portal 484 also permits more general patient management via its communication (e.g. wireless, cloud) with the stimulation generator 424, patient control 438, and/or app 482 on mobile device 480. In some examples, the portal 484 may be implemented via at least some of substantially the same features and attributes as portal 3082 of FIG. 9E and/or portal 3036 of FIG. 9C in association with FIGS. 9A-9E.

The stimulation generator 424 is not limited to these features and capabilities and, it will be understood, that in other examples, additional other features and capabilities 468 may be available.

Building on the foregoing examples, FIGS. 6B-6E are graphs illustrating example patterns for patients titrating stimulation amplitudes, such as during an initial period of therapy use of an IMD (e.g., for sleep disordered breathing). Determination of the information in, and/or use of, these graphs may comprise an example method and/or device, which in turn may comprise an example implementation of, and/or at least some of substantially the same features and attributes as, the examples of FIGS. 1A-6A.

The graphs of FIGS. 6B-6E each illustrate stimulation amplitude changes made by (or for) a patient, as represented via line 503, and a reference by which attention-warranting-titration patients may be identified. On the graphs in FIGS. 6B-6E, the Y-axis represents the stimulation amplitude change (e.g., from the baseline illustrated as 0.0 Volts and which may extend up to 1.2 Volts in some examples) and the X-axis represents the number of usage days since first use. In some examples, each stepwise change in stimulation may include 0.1 Volts.

In some examples, a titration reference (which may sometimes interchangeably be referred to herein as “a stimulation amplitude change reference”) may be represented by line 501 and may comprise (or alternatively may be defined relative to) an average (e.g., median, mean) of stimulation amplitude changes made by a plurality of compliant patients over the period of initial use. In some examples, the titration reference may comprise (or alternatively may be defined relative to) a standard deviation from the median/mean (line 501), which is represented via shaded region 505. However, in some examples, the titration reference (e.g. shaded region 505) may comprise (or be defined relative to) a confidence interval and/or a pre-selected range based on predetermined titration guidelines.

As shown by each of FIGS. 6B-6E, each attention-warranting-titration patient exhibits some stimulation amplitude changes, illustrated via line 503, which deviate from titration reference when implemented as line 501. For instance, in the example of FIG. 6B, the stimulation amplitude (line 503), which may be patient selected, rapidly and significantly deviates from the reference (e.g., line 501) within the first 5-10 days of use, and still differs from the reference (line 501) up through day 20. Among other potential factors, this deviation may be quantified as a percentage difference (or other metric) from the reference (line 501) and upon the deviation meeting a criteria (e.g., exceeding a threshold deviation), the patient may be flagged as a patient which is non-compliant or likely to become non-compliant due to overly-aggressively titrating, e.g. overly-aggressively increasing their stimulation amplitude within a given time frame (e.g., 5, 10, or 20 days).

Similarly, in the example of FIG. 6C, the stimulation amplitude (line 503), which may be patient selected, rapidly and significantly deviates from (e.g., is much higher than) the reference (e.g. line 501) within the first 5-10 days of use, and still differs from the reference (line 501) up through about day 50, and again from about day 50 through the end of the initial use period (e.g., 90 days). As in the example FIG. 6B, among other potential factors, this deviation shown in FIG. 6C may be quantified as a percentage difference (or other metric) from the reference (line 501) and upon the deviation meeting a criteria (e.g., exceeding a threshold deviation), the patient may be flagged as a patient which is non-compliant or likely to become non-compliant due to overly-aggressively titrating, e.g. overly-aggressively increasing their stimulation amplitude within a given time frame (e.g., 5, 10, 20, 50, 90 days).

Moreover, even though the patient represented in FIG. 6D exhibits a stimulation amplitude change which is static from about 20 days to 90 days, the rapid increase over the first 20 days (and attendant significant deviation from line 3001), in some examples, may still provide a basis to identify (e.g., flag) this patient as being non-compliant and/or likely to become non-compliant during or after the initial use period.

Finally, the patient represented in FIG. 6E exhibits stimulation amplitude changes which generally correspond to the reference (implemented as or relative to line 501 in one example) such that the patient may be considered compliant and/or likely to remain compliant over a longer term. However, during the period of about 30 days to 35 days, the patient exhibits rapid increases in stimulation amplitude changes, which may provide a basis to identify (e.g., flag) this patient as being non-compliant and/or likely to become non-compliant during or after the initial use period, even with the later largely static period from day 40 through 90 days. It is also recognized, in some examples, that increases or decreases in stimulation amplitude changes could be attention-warranting. Increases in stimulation amplitude could result in risk of non-compliance and decreases in stimulation amplitude could result in risk of sub-therapeutic or non-therapeutic stimulation.

Accordingly, in some examples, one or more of the patients represented in FIGS. 6B-6E may identified as an attention-warranting titration patient to be brought to the attention of the clinician so that some counsel or intervention can be made during the period of deviation from a titration reference (e.g., line 501). This early notification to the clinician may result in intervention (e.g., limiting patient changes to stimulation amplitude, related counseling, etc.) with the aim of increasing the likelihood of compliance in both the short term and long term, which in turn may improve patient outcomes (e.g., disease burden decrease) and/or patient usage (e.g., maintain minimum usage for a number of hours-per-day, number of days-per-week, number of weeks, etc.) in both the short term and long term.

While the examples of FIGS. 6B-6E are primarily directed to patient titration during an initial use period (e.g., 90 days in some examples) of a therapeutic stimulation device, it will be understood that the features and attributes of these examples (in context with all the example of the present disclosure) may be implemented for periods of use beyond the initial use period.

With further reference to the input portion 401 as shown in FIG. 6A, in some examples, a tracked patient metric (e.g. 112 in FIG. 3) can include a therapy outcome parameter 412 which may track one or more therapy outcomes. In some examples, the therapy outcome parameter 412 may comprise objective parameters 414 and in others, the outcome may comprise subjective parameters 416. Example therapy outcomes can include any metric shown to improve health or a condition resulting from the stimulation therapy, any metric corresponding to the patient's use of, or adherence to, the stimulation therapy, and/or any other metric contributing to the well-being of the patient, but are not limited to, an average apnea frequency (e.g. AHI) per sleep treatment period; Epworth Sleepiness Scale (ESS), snoring; arousals; and/or patient perceived sleep quality. In one example, an objective therapy outcome 414 comprises a deviation from a threshold in average apnea frequency (e.g. AHI) per sleep treatment period. In another example, one of the objective therapy outcomes 414 includes a threshold maximum decrease in average apnea frequency (e.g. AHI) per sleep treatment period from a baseline average apnea frequency per sleep treatment period. As previously noted, the examples of FIGS. 2-5 provided a graphic representation of at least some objective therapy outcomes.

With continued reference to input portion 401 of FIG. 6A, a tracked patient metric (e.g. 112 in FIG. 3) may, in some examples, further include parameters indicating an amount the stimulation therapy is used. In various examples, “used” means the stimulation therapy in an “on” state in which stimulation therapy is being delivered. In some such examples, the stimulation therapy may include a closed loop mode and/or an open loop mode, as further described below in association with at least FIGS. 8A, 9A.

In various examples, the tracked patient metric (e.g. 112 in FIG. 3) includes a usage parameter 418 of the stimulation therapy. In the illustrated example of at least FIGS. 4-5, usage may be evaluated as a percentage of usage during the sleep treatment period as compared to a target amount of usage (e.g. maximum target usage, in some examples) during the sleep treatment period. In some examples, the target amount of usage may comprise a number of hours (or partial hours) of usage (e.g. parameter 420 in FIG. 6A) that preferably generally matches the number of hours (or partial hours) of the sleep treatment period. In some examples, usage information may be tracked on a number of hours used on a per night basis over a plurality of nights. In some examples, per parameter 422 in input portion 401 of FIG. 6A, usage information may be tracked on a number of nights used (for at least a selectable predetermined number of hours usage per night, e.g. 4 hours in some examples) within a selectable time frame such as, but not limited to, a per week basis, per month basis, per 90 day window basis, and so on. In some examples, the target usage parameter includes a target percentage of a maximum usage of the pulse generator (e.g. implantable). In one example, the maximum usage is defined as a number of hours of stimulation therapy for each of the multiple number of sleep treatment periods. In one example, the number of hours is four, however, this number of hours could be between 3-4 hours or between 4-5 hours, or another number of hours.

In another example, the target usage parameter comprises a number of nights (e.g. 422 in FIG. 6A) of the multiple number of sleep treatment periods for which a minimum number hours (e.g. 420 in FIG. 6A) of stimulation therapy was delivered. Generally, in some examples, the usage parameter may include quantifying an amount of time the pulse generator (e.g. implantable) has applied stimulation over one or more number of nightly sleep treatment periods.

Stimulation can be applied to target tissue(s) comprising muscles and/or nerves innervating such muscles.

FIG. 6F illustrates a simplified patient anatomy 600 including nerves and/or muscles that can be target tissues. Among other nerves, FIG. 6F illustrates an infrahyoid muscle (IHM)-innervating nerve and associated muscles. In some examples, an IHM-innervating nerve may comprise a nerve or nerve branch which innervates (directly or indirectly) at least one infrahyoid muscle, which may sometimes be referred to as an infrahyoid strap muscle. In some examples, IHM-innervating nerves/nerve branches extend from (e.g. originates) from a nerve loop called the ansa cervicalis (AC) or the “AC nerve loop”, which stems from the cervical plexus, e.g. extending from cranial nerves C1-C3. Accordingly, in some examples, at least some IHM-innervating nerves may correspond to an ansa cervicalis (AC)-related nerve in the sense that such nerves/nerve branches (e.g. IHM-innervating nerves) do not form the AC nerve loop but extend from the AC nerve loop. At least because the AC nerve loop is the origin for some nerves which innervate muscles other than the infrahyoid muscles, some AC-related nerves do not comprise IHM-innervating nerves. Moreover, it will be understood that in some examples, stimulation applied to a portion (e.g., superior root) of the AC nerve loop (and/or to nerves from which the AC nerve loop originates) may activate IHM-innervating nerves/nerve branches, which extend from the AC nerve loop. However, implementing stimulation (e.g. to influence upper airway patency) occurring at more proximal locations, such as along the superior root of the AC nerve loop may be more complex because of the number/type of different nerves and number/type of different muscles innervated via a superior root of the AC nerve loop such that selective activation of a particular infrahyoid muscle (via stimulation along the superior root) may be quite challenging in some circumstances.

For example, stimulation can be applied at target location A, target location B, and/or target location C. Portion 629A of the AC-main nerve 615 (e.g. a portion or trunk connecting to the AC nerve loop 619) extends anteriorly from a first cranial nerve C1 with a segment 617 running alongside the hypoglossal nerve 635 (target location A) until the AC-main nerve 615 diverges from the hypoglossal nerve 635 to form a superior root 625, which forms part of an AC nerve loop 619. Target location B may be located at superior root 625. A portion of the hypoglossal nerve 635 extends distally to innervate the genioglossus muscle 604. The superior root 625 extends inferiorly until reaching near bottom portion 618 of the AC nerve loop 619, from which the nerve loop 619 further extends superiorly to form a lesser root 627 (i.e. the inferior root of loop 619) to complete the AC nerve loop 619, and with portions 629B and 629C joining the second and third cranial nerves, C2 and C3, respectively.

Several branches 631 extend off the AC nerve loop 619, including branch 642 (which includes target location C) which innervates the sternothyroid muscle (STM) 644 and a portion of the sternohyoid muscle (SHM), e.g., SHM inferior. Another branch 652, near bottom portion 618 of the AC nerve loop 619, innervates another portion of the SHM, e.g., SHM superior 654. The branches 631 further include branch 632 which innervates the omohyoid muscle (OHM) 634. The AC-related nerve 614 may include additional branches (beyond those illustrated and described) extending from the AC nerve loop 619. In some examples, the collective arrangement of the AC-main nerve 615 (including at least superior root 625 of the AC nerve loop 619) and its related branches (e.g. at least 632, 642, 652) when considered together, or any of those elements individually, may sometimes be referred to as an IHM-innervating nerve 616. It will be further understood that at least one such IHM-innervating nerve 616 is present on both sides (e.g. right and left) of the patient's body.

In some examples, target location A is located where segment 617 runs alongside the hypoglossal nerve 635 and innervates at least the genioglossus muscle 604. In some such examples, these more proximal portions of the hypoglossal nerve 635 and the AC-main nerve 615 may be activated, such as via selective stimulating at least the nerve fibers (e.g. fascicles) of the hypoglossal nerve 635 responsible for protrusion of the tongue (via activation of the genioglossus muscle) and of the AC-main nerve 615 responsible for activation of at least the sternothyroid muscle, in some examples.

In some examples, target location B is located along the superior root 625 and provides a more proximal location from which at least some branches (e.g. 642, 652) may be activated, such as via selective stimulation of nerve fibers within the superior root 625. In some examples, target location C is located along branch 642, which extends distally from a superior root 625 of the AC nerve loop 619 and innervates at least the STM 644. Accordingly, at least branch 642 may sometimes be referred to as an IHM-innervating nerve. Stimulating at the target location C may be used to completely capture and/or fully activate the STM 644 and/or to promote upper airway patency. In some examples, target location C of the IHM-innervating nerve 616 may innervate the STM 644 and the SHM inferior. Stimulation at target location C may thereby fully activate the STM 644 to pull the thyroid cartilage inferiorly, via branch 645A, and, optionally activate the SHM inferior to pull the hyoid bone inferiorly, via branch 645B. Activating the SHM superior 654 alone or activating a combination of the SHM superior 654 and SHM inferior (STM 644) may have greater impact on hyoid bone movement (inferiorly) than activation of the SHM inferior without activating the SHM superior 654. As such, in some examples, activating the SHM inferior may have minimal (or below a threshold) impact on the movement of the hyoid bone. While stimulation of just the hypoglossal nerve 635 (or some branches thereof) may be effective in increasing upper airway patency to a sufficient degree to ameliorate OSA in high percentage of qualified patients (e.g., least about 70 to 80 % in some examples) when using certain types of implantable neurostimulation devices, some patients may benefit from stimulation of the an IHM-innervating nerve (e.g., 642, 619) in addition to, or instead of, stimulation of the hypoglossal nerve 635. Moreover, for a particular patient, certain positions of the head-and-neck and/or of their body (e.g., supine, lateral decubitis, etc.) may be treated more effectively by stimulating an IHM-innervating nerve (e.g., more direct via branch 642 or less directly via AC nerve loop 619), with or without stimulation of the hypoglossal nerve 635.

While noted elsewhere, in some examples stimulation may be applied at locations more distal than location C, such as applying stimulation in close proximity to the particular target IHM (e.g. STM 644). In some such examples, stimulation may be applied at the IHM itself and/or at a neuromuscular junction of the target IHM and the nerve branch directly innervating the target IHM.

Referring now in addition to FIG. 7, which illustrates a block diagram schematically representing an example sensing portion 2000. In some examples, an example method may employ and/or an example SDB care device may comprise the sensing portion 2000 to sense physiologic information and/or other information, with such sensed information relating to care of a wide variety of physical conditions such as, but not limited to, sleep disordered breathing care, pelvic care, cardiac care, among other uses.

The sensed information may be used to implement at least some of the example methods and/or examples devices described in association with at least FIGS. 1A-7 and/or FIGS. 9A-9E. In particular, the sensed information obtained and/or received via sensing portion 2000 of FIG. 7 may be used as the physiologic parameter/source 404 of information in source portion 402 of input portion 401, which provides inputs to stimulation generator 424 in FIG. 6A for guiding stimulation therapy per a patient metric according to example methods and/or example devices of the present disclosure.

It will be understood that the sensing portion 2000 may be implemented as a single sensor or multiple sensors, and may comprise a single type of sensing or multiple types of sensing. In addition, it will be further understood that the various types of sensing schematically represented in FIG. 7 may correspond to a sensor and/or a sensing modality.

In some examples, the sensed information may refer to physiologic signals (e.g. biosignals) and/or metrics which may be derived from such physiologic signals. For example, among other sensed physiologic signals, one physiologic signal may comprise respiration (parameter 2005 in FIG. 7), from which various metrics may be derived such as, but not limited to, respiratory rate, respiratory rate variability, respiratory phase, rate times volume, waveform morphology, and more. The respiration information may be sensed via one or more sensing modalities described below (and/or other sensing modalities) such as, but not limited to, accelerometer 2026, electrocardiogram (ECG) 2020, impedance 2036, pressure 2037, temperature 2038, acoustic 2039, and/or other sensing modalities, at least some of which are further described below. The respiration information may be used for a wide variety of purposes such as, but not limited to, timing stimulation relative to respiration, disease burden, sleep-wake status, arousals, etc. In some such examples, the detection of disease burden may comprise detection of sleep disordered breathing events, which may be used in determining, assessing, etc. therapy outcomes such as, but not limited to, AHI.

In some examples, the sensed physiologic information may comprise cardiac information (2006) obtained from a cardiac signal and from which various metrics may be derived such as, but not limited to, heart rate (HR), heart rate variability (HRV), P-R intervals, waveform morphology, and more. One example of a cardiac signal many comprise an ECG signal, as represented at 2020 in FIG. 7. Accordingly, the cardiac information and/or signal may be sensed via one or more sensing modalities further described below (and/or other sensing modalities) such as, but not limited to, cardiac sensor 2023, accelerometer 2026, ECG 2020, electromyogram (EMG) 2022, impedance 2036, pressure 2037, temperature 2038, and/or acoustic 2039. In some examples, the sensed physiologic information (e.g. via sensing portion 2000) may comprise a wide variety of physiologic information other (2007) than respiration and/or cardiac information, with at least some examples further described below in association with FIG. 7, and other examples throughout the present disclosure.

The sensed physiologic signals and/or information (e.g. respiration 2005, cardiac 2006, and/or other information 2007) may be used for a wide variety of purposes such as, but not limited to, determining sleep-wake status (e.g. various sleep onset determinations), timing stimulation relative to respiration, determining disease burden, determining arousals, etc. In some such examples, the determination of disease burden may comprise detection of sleep disordered breathing events, which may be used in determining, assessing, etc. therapy outcomes such as, but not limited to, AHI, as well as titrating stimulation parameters, adjusting sensitivity of sensing the physiologic information, etc.

For instance, in one non-limiting example, an electrocardiogram (ECG) sensor 2020 in FIG. 7 may comprise a sensing element (e.g. electrode) or multiple sensing elements arranged relative to a patient's body (e.g. implanted in the transthoracic region) to obtain ECG information. In some examples, the ECG information may comprise one example implementation to obtain cardiac information, including but not limited to, heart rate 2025A (HR), heart rate variability 2025B (HRV) and other cardiac parameters 2025C, which may be used (with or without other information) in determining delivering stimulation therapy and associated sensing (e.g. inputs) for determining effectiveness of the therapy and/or implementing the therapy, as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g. outer housing) of an implantable pulse generator (IPG), such as further described later in association with at least FIG. 9C. In such instances, other ECG electrodes are spaced apart from the ECG electrode associated with the IPG. In some examples, at least some ECG sensing electrodes also may be employed to deliver stimulation to a nerve or muscle, such as but not limited to, an upper airway patency-related nerve (e.g. hypoglossal nerve) or other nerves or muscles.

In some examples, other types of sensing may be employed to obtain cardiac information (including but not limited to heart rate and/or heart rate variability), such as a cardiac sensor 2023 shown in FIG. 7, which may comprise one or more of a ballistocardiogram sensor(s), seismocardiogram sensor(s), and/or accelerocardiogram sensor(s). In some examples, such sensing is based on and/or implemented via accelerometer-based sensing such as further described below in association with accelerometer 2026.

In one aspect, in some examples in which the cardiac sensor 2023 comprises a ballistocardiogram sensor, the sensor senses cardiac information caused by cardiac output, such as the forceful ejection of blood from the heart into the great arteries that occurs with each heartbeat. The sensed ballistocardiogram information may comprise heart rate (HR) 2025A, heart rate variability (HRV) 2025B, and/or additional cardiac morphology 2025C. In some examples such ballistocardiogram-type information may be sensed from within a blood vessel in which the sensor (e.g. accelerometer) senses the movement of the vessel wall caused by pulsations of blood moving through the vessel with each heartbeat. This phenomenon may sometimes be referred to as arterial motion.

In some examples in which the cardiac sensor 2023 comprises a seismocardiogram sensor, the sensor 2023 may provide cardiac information which is similar to that described for ballistocardiogram sensor, except for being obtained via sensing vibrations, per an accelerometer (e.g. single or multi-axis), in or along the chest wall caused by cardiac output. In particular, the seismocardiogram measures the compression waves generated by the heart (e.g. per heart wall motion and/or blood flow) during its movement and transmitted to the chest wall. Accordingly, the sensor 2023 may be placed in the chest wall.

In some such examples of sensing per sensor 2023, such methods and/or devices also may comprise sensing a respiratory rate and/or other respiratory information.

In some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-neck region otherwise suitable to sense EEG information. Accordingly, the EEG sensor(s) 210 are located near the brain and may detect frequencies associated with electrical brain activity.

In some examples, a sensing element used to sense EEG information is chronically implantable, such as in a subdermal location (e.g. subcutaneous location external to the cranium skull), rather than an intracranial position (e.g. interior to the cranium skull). In some examples, the EEG sensing element is placed and/or designed to sense EEG information without stimulating a vagus nerve at least because stimulating the vagus nerve may exacerbate sleep apnea, particularly with regard to obstructive sleep apnea. Similarly, the EEG sensing element may be used in a device in which a stimulation element delivers stimulation to a hypoglossal nerve or other upper airway patency-related nerve without stimulating the vagus nerve in order to avoid exacerbating the obstructive sleep apnea.

In some examples, sensed EEG information may be used as part of (or solely in) making a sleep-wake determination, such as sleep onset, and wake onset. Among other uses, this sleep-wake information may help provide overall sleep hours, which may comprise part of therapy outcome, in some examples.

In some examples, sensed EEG information may be used to detect sleep stages during sleep. Among other uses, this sensed sleep stage may help determine an absolute amount or relative amount of deep sleep, REM sleep per night, and/or other sleep metrics. For instance, such information may be used to evaluate whether a particular stimulation solution setting corresponds to a patient's most therapeutic stimulation energy settings/parameters based on (at least or in part) the recognition more deep sleep typically corresponds to the most or more therapeutic stimulation energy settings whereas less deep sleep typically corresponds to lesser therapeutic stimulation energy settings.

In some examples, sensed EEG information may be used to detect arousals, which may comprise one aspect of determining therapy outcome. Among other uses, the detection of more arousals may provide an indication of the patient exhibiting more daytime sleepiness, which in turn may lead to adjustments to stimulation solution settings (e.g. values of stimulation energy parameters) in order to minimize arousals.

In some examples, the above-described aspects regarding the use of sensed EEG information may be combined in whole, or part, to provide an overall sleep efficiency parameter. In some such examples, the sleep efficiency parameter may be based on: 1) sleep duration; 2) sleep depth; and/or 3) events (e.g. number of arousals). In some examples, the sleep efficiency parameter may be compared to a reference sleep efficiency parameter such as (but not limited to): 1) a reference sleep duration (e.g. 8-9 hours); 2) a reference sleep depth (e.g. a minimum duration of deep sleep and REM sleep; and/or 3) few or no arousals.

In some examples, the sensed EEG information and/or information derived therefrom (e.g. sleep efficiency parameter) may comprise at least a portion of patient metrics tracked, etc. via the patient metric engine 2916 as part of care engine 2900 described later in association with FIG. 9A.

In some examples the sensing portion 2000 may comprise an electromyogram (EMG) sensor 2022 to obtain and track EMG information. In some such examples, the EMG sensor may comprise an electrode positioned near the tongue to detect signals indicative of voluntary control of the tongue, which in turn may be indicative of wakefulness. In some examples, the sensed EMG signals may be used to identify sleep and/or obstructive events. In some examples, the EMG sensor 2022 also can be placed at other locations within (or on) the body to detect muscle activity which may be indicative of sleep parameter such as, but not limited to, sleep depth. In some such examples, one sleep depth parameter may comprise REM sleep, which may be used to determine whether a target amount (e.g. minimum) of REM sleep occurred. In some examples, the detected EMG information may be used to detect arousals and/or overall patient movement. These examples of determining and/or using sensed EMG information may be used as part of determining patient metrics (e.g. therapy outcome, usage, other) by which stimulation energy parameters may be determined, adjusted, etc. in order to maintain and/or improve those patient metrics according to various examples of the present disclosure.

In some examples, any one or a combination of the various sensing modalities (e.g. EEG, EMG, etc.) described in association with FIG. 7 may be implemented via a single sensing element 2014.

In some examples, the sensing portion 2000 may comprise an accelerometer 2026. In some examples, the accelerometer 2026 and associated sensing (e.g. motion at (or of) the chest, neck, and/or head, respiratory, cardiac, posture, etc.) may be implemented according to at least some of substantially the same features and attributes as described in Dieken et al., ACCELEROMETER-BASED SENSING FOR SLEEP DISORDERED BREATHING (SDB) CARE, published as U.S. Publication No. 2019-0160282 on May 30, 2019, and PCT Publication No. WO 2022/020489, published on Jan. 27, 2022, and entitled “DISEASE BURDEN INDICATION”; and PCT Publication No. WO2022/261311, published on Dec. 15, 2022, and entitled “RESPIRATION SENSING”, and which are incorporated by reference herein in their entirety. In some examples, the accelerometer may comprise a single axis accelerometer while in some examples, the accelerometer may comprise a multiple axis accelerometer.

Among other types and/or ways of sensing information, the accelerometer sensor(s) 2026 may be employed to sense or obtain a ballistocardiogram, a seismocardiogram, and/or an accelerocardiogram (see cardiac sensor 2023 and related disclosure), which may be used to sense (at least) heart rate 2025A and/or heart rate variability 2025B (among other information such as respiratory rate in in some instances), which may be used as part of determining respiratory information, cardiac information, as described throughout the examples of the present disclosure. In some examples, this sensed information also may be used in determining sleep-wake status.

In some examples, the accelerometer 2026 may be used to sense activity, posture, and/or body position as part of determining a patient metric, the sensed activity, posture, and/or body position may sometimes be at least partially indicative of a sleep-wake status, which may be used as part of automatically initiating, pausing, and/or terminating stimulation therapy.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g. electrodes) spaced apart from each other across a portion of the patient's body. In some such examples, one of the sensing elements may be mounted on or form part of an outer surface (e.g. case) of an implantable pulse generator (IPG) (see 3025 in FIG. 9C) or other implantable sensing monitor, while other sensing elements may be located at a spaced distance from the sensing element of the IPG or sensing monitor. In at least some such examples, the impedance sensing arrangement integrates all the motion/change of the body (e.g. such as respiratory effort, cardiac motion, etc.) between the sense electrodes (including the case of the IPG when present). Some example implementations of the impedance measurement circuit will include separate drive and measure electrodes to control for electrode to tissue access impedance at the driving nodes.

In some examples, the sensing portion 2000 may comprise a pressure sensor 2037, which senses respiratory information, such as but not limited to respiratory cyclical information. In some examples, the pressure sensor 2037 may be located in direct or indirect continuity with respiratory organs or airway or tissues supporting the respiratory organs or airway in order to sense respiratory information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiratory information, snoring, etc.

In some examples, sensing portion 2000 may comprise body motion parameter 2035 by which patient body motion may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities, as described throughout various examples of the present disclosure.

In some examples, the sensing portion 2000 in FIG. 7 may comprise a body position/posture parameter 2042 and/or body motion parameter 2035 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine a patient metric. As described elsewhere herein, in some examples posture may be considered as one of several parameters when determining a probability of sleep (or awake). In some such examples, the sleep-wake status may be used to initiate, pause, and/or terminate stimulation therapy within a nightly treatment period.

In addition or alternatively, sensing activity, motion, and/or body position (e.g. posture) may be used to track a relative degree to which a patient is more active or less active during daytime hours, which may comprise one objective measure of therapy outcome because if the patient is sleeping better at night due to a desirable stimulation solution settings (e.g. values of stimulation energy parameters) which better control sleep disordered breathing, the patient may be much more active during daytime (non-sleep) hours as compared to a baseline in which their sleep disordered breathing was poorly controlled (corresponding to inferior stimulation energy settings) or not controlled at all. Similarly, sensing activity and/or motion as described herein also may be used to detect if the patient tends to falls asleep during daytime (e.g. non-sleep) hours, which may be an objective therapy outcome parameter by which stimulation energy parameters (and associated usage, and other therapy outcome parameters) may be evaluated and potentially adjusted according to at least some examples of the present disclosure. This objective therapy outcome information also may be used in conjunction with subjective therapy outcome information such as, but not limited to, the Epworth Sleepiness Scale (ESS) (via 416 in FIG. 6A) and/or other forms of patient input (410 in FIG. 6A) regarding the patient's perceived daytime sleepiness, daytime functional ability, perceived sleep quality, etc. This objective and subjective information may be used as part of the inputs received by stimulation generator 424 from input portion 401 in the device 400 of FIG. 6A, which may support or enable the examples of FIGS. 1A-5, 7 of guiding therapy per a patient metric.

For instance, sensing an upright posture typically is associated with a wakeful state, such as standing or walking. However, as noted elsewhere, a person could be in an upright sitting position and still be in a sleep state (e.g. sleeping in a chair). Accordingly, posture may be just one parameter used in determining a sleep-wake state, along with at least some other parameters described in association with sensing portion 2000 of FIG. 7 and/or care engine 2900 in FIG. 9A. Conversely, sensing a supine or lateral decubitis (i.e. laying on a side) posture typically is associated with a sleep state. However, a patient might be in such a position without being asleep, such that other parameters in addition to, or instead of, posture may significantly enhance determination of sleep-wake status.

Moreover, sensing posture may not be limited to sensing a static posture but extend to sensing simple changes in posture (or body position), which may be indicative of a sleep-wake state at least because certain changes in posture (e.g. from supine to upright) are mostly likely indicative of a wake state. Similarly, more complex or frequent changes in posture and/or body position may be further indicative of a wake state, whereas maintaining a single stable posture for an extended period time may be indicative of a sleep state.

In some examples, the sensing portion 2000 can comprise an other parameter 2041 to direct sensing of, and/or receive, track, evaluate, etc. sensed information other than the previously described information sensed via the sensing portion 2000.

As further shown in FIG. 7, in some examples the sensing portion 2000 may comprise a temperature sensor 2038. In some examples, such sensed temperature may be tracked, evaluated, etc. in association with patient metric 2916 in care engine 2900 in FIG. 9A.

In some examples, the sensed temperature may be used as one factor in making a sleep-wake status determination according to the examples of the present disclosure. In one aspect, the temperature sensor 2038 may sense and track a patient's normal fluctuation (e.g. temperature profile) in body temperature within a 24 hour daily period, which may exhibit on the order of a 2 degree F change. For most patients, their body temperature may reach and remain at the high end (e.g. 99.5 F) of its range during the middle of the day and evening (e.g. 7 pm) before falling throughout late evening and overnight to the low end (e.g. 97.5 F) of its range by early morning (e.g. 5 or 6 am). In some examples, the temperature sensor 2038 may sense a change in the sensed temperature which occurs within a selectable time window of a 24 hour period and which exceeds a selectable threshold. In some examples, the selectable time window may comprise one hour, two hours, or other time periods. In some such examples, one method comprises selecting that a change of a predetermined number of degrees within the selectable time window will correspond to either a wake-to-sleep state transition or a sleep-to-wake state transition.

In some example methods, sensing a change in temperature (such as via sensor 2038) during a treatment period may be used to identify sleep disordered breathing behavior. In some such examples, additional sensed information (as described in examples of the present disclosure) may be used in addition to sensed temperature to identify sleep disordered breathing (SDB) behavior.

In some examples, this temperature fluctuation information sensed via temperature sensor 2038 may be used to automatically implement a boundary or limit on the beginning and end of the treatment period, such that the lowest sensed body temperature may be used to at least partially mark a boundary of an end of a treatment period for a typical patient which sleeps at night. Similarly, the highest sensed body temperature (e.g. held for an extended period) may be used to at least partially implement a boundary at a beginning of a treatment period.

In some examples, these same temperature-based boundaries may be used as one factor (among other factors) to determine a sleep-wake status. At least some other factors, which may be used with this sensed temperature fluctuation information to determine a sleep-wake status, may comprise a time of day parameter, accelerometer information, cardiac information, respiratory information, etc.

In some examples, smaller yet detectable temperature changes within a treatment period may be used to at least partially determine a patient metric. For instance, a detectable temperature change may be sensed as a result of patient exertion to breathe in response to an apnea event, given the greater muscular effort in attempting to breathe.

Moreover, in some examples, such sensed temperature fluctuation information may provide a more distinctive or characteristic indication of a sleep or wake period when compared with heart rate or body position, which may exhibit more changes, some of which are not necessarily indicative of a sleep period or wake period, at least in some instances.

In some examples, at least some of the sensors and/or sensor modalities described in association with FIG. 7 (and/or FIG. 9A) may be incorporated within or on a pulse generator (PG 3025 in FIG. 9C), or within or on a microstimulator (e.g. generally, device 3060), which may be at least partially implantable in some examples.

FIG. 8A is a block diagram schematically representing an example stimulation portion 2200. In some examples, the stimulation portion 2200 may comprise an example further implementation of, and/or at least some of substantially the same features and attributes as, the stimulation generator 424 of FIG. 6A, the control portion (e.g. FIG. 1C, FIGS. 9A-9E) of the present disclosure. Accordingly, the various functions and parameters of the stimulation portion 2200 may be implemented in a manner supportive of, and/or complementary with, the various functions, parameters, portions, etc. of device 400 of FIG. 6A (including stimulation generator 424) and/or various functions, parameters, portions, etc. relating to stimulation throughout examples of the present disclosure.

In some examples, via target tissue parameter 2210, stimulation may be delivered to selectable target tissues such as, but not limited to, upper airway patency-related tissues. In some examples, the upper airway patency-related tissue may comprise a hypoglossal nerve and/or muscle (e.g. genioglossus muscle) innervated by the hypoglossal nerve to cause contraction of at least the protrusor muscles to cause protrusion of the tongue to increase and/or maintain upper airway patency. In some examples, the upper airway patency-related tissue may comprise infrahyoid muscle-related nerves which includes a nerve(s) innervating one or more infrahyoid strap muscles (thyrohyoid, omohyoid, sternohyoid, and/or sternothyroid), and may include an ansa cervicalis-related nerve. The infrahyoid muscle-related nerves may sometimes be referred to as infrahyoid muscle-innervating nerves. In one example, target tissues may include any other muscles which affect and/or promote upper airway patency, and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different nerves for stimulation, the target tissue parameter 2210 also may comprise adjusting stimulation parameters via selecting between (or using a combination of) various locations along a nerve such as stimulating multiple different sites along a particular nerve, with some stimulation sites being more distal and some being more proximal. For example, at least some target locations A, B, and C shown in FIG. 6F.

In some examples, in addition to or instead of selecting different nerves for stimulation, the target tissue parameter 2210 also may comprise adjusting stimulation parameters via selecting between (or using a combination of) different fascicles within a particular nerve in order to selectively stimulate target motor fibers while omitting (or minimally impacting) stimulation of other, non-target motor fibers and/or to selectively stimulate target motor fibers while omitting (or minimally impacting) stimulation of other, non-target sensory fibers.

In some examples, the stimulation portion 2200 may implement stimulation according to a bilateral parameter 2212 in which stimulation is applied to a target tissue on both sides (e.g. left and right) of the patient's body. In some such examples, this bilateral stimulation may be delivered to the same nerve (e.g. hypoglossal nerve) on both sides of the body. However, in some examples, the bilateral stimulation may be delivered to different nerves (e.g. hypoglossal nerve and infrahyoid muscle-innervating nerve) such as stimulating one nerve (e.g. hypoglossal nerve) on a left side of the body while stimulating another nerve (e.g. infrahyoid muscle-innervating nerve) on a right side of the body, or vice versa.

In some examples, the bilateral parameter 2212 may be implemented in a manner complementary with the alternating parameter 2232, simultaneous parameter 2234, or demand parameter 2236 of multiple function 2230, as further described below.

In some examples, the stimulation portion 2200 may comprise a multiple function 2230 by which various stimulation parameters may be implemented in dynamic arrangements. In some such examples, the stimulation portion 2200 may comprise an alternating parameter 2232 by which stimulation of one target tissue (e.g. hypoglossal nerve) may be alternated with stimulation of at least one other target tissue (e.g. infrahyoid muscle-innervating nerve). However, the alternating parameter 2232 also may be applied in combination with the bilateral parameter 2212 to apply stimulation to the same nerve (or different nerves) on opposite sides of the body in which stimulation may be applied on a left side of the body and then applied on the right side of the body in an alternating manner.

In some examples, the stimulation portion 2200 may comprise a simultaneous parameter 2234 by which stimulation may be applied simultaneously to at least two different target tissues. In some examples, the at least two different target tissues comprise two different nerves, such as the hypoglossal nerve and an infrahyoid muscle-innervating nerve. However, in some examples, the at least two different target tissues may comprise two different locations along the same nerve or two different fascicles of the same nerve. In some examples, the simultaneous parameter 2234 may apply stimulation per bilateral parameter 2212 simultaneously on opposite sides of the body to the same nerve (e.g. hypoglossal nerve) or different nerves.

In some examples, the stimulation portion 2200 may comprise a demand parameter 2236 by which stimulation may be applied to one or more nerves on a demand basis. For example, stimulation may be applied to one nerve (e.g. hypoglossal nerve) which may be sufficient to achieve the patient metric (e.g. therapy outcome and/or usage) for most nights, for most sleeping positions (e.g. left and right lateral decubitis, prone), etc. but may become insufficient for some nights (e.g. after consuming alcohol or certain drugs which relax upper airway muscles), some sleeping positions (e.g. supine). In the latter situation, in order to achieve the target patient metric, via the demand parameter 2236, stimulation of a different nerve (e.g. infrahyoid muscle-innervating nerve) may be implemented in addition to, or instead of, stimulation of the first nerve (e.g. hypoglossal nerve) which was previously being stimulated. In some examples, the first or primary nerve being stimulated may be a nerve other than the hypoglossal nerve such as, but not limited to, the infrahyoid muscle-innervating nerve.

In some examples, the stimulation portion 2200 also may further implement at least some aspects of the stimulation generator 424 (FIG. 6A) and/or the parameters 2210, 2212, 2230 of stimulation portion 2200 according to one or more of a closed loop parameter 2220, open loop parameter 2222, and nightly titration parameter 2224.

In some examples, the stimulation portion 2200 comprises a closed loop parameter 2220 to deliver stimulation therapy based on sensed patient physiologic information and/or other information (e.g. environmental, temporal, etc.). In some such examples, via the closed loop parameter 2220 the sensed information may be used to control the particular timing of the stimulation according to respiratory information, in which the stimulation pulses are triggered by or synchronized with specific portions (e.g. inspiratory phase) of the patient's respiratory cycle(s). In some such examples and as previously described, this respiratory information and/or other information used with the closed loop parameter 2220 may be determined via the sensors, sensing elements, devices, sensing portions, as previously described in association with at least FIG. 7.

In some examples, with or without timing stimulation relative to sensed respiratory information, the closed loop mode (2220) may comprise delivering stimulation therapy in response to sensed disease burden, such as the average number of apnea events per a time period, such as an apnea-hypopnea index (AHI) of average number of apnea events per hour. For example, for some periods of time within a nightly treatment period or over the course of several days/weeks, a patient may experience few sleep disordered breathing events (e.g. apnea events), such that stimulation therapy may be not delivered. However, upon the patient beginning to experience sleep disordered breathing at a level high enough to warrant stimulation therapy, then via the closed loop parameter 2220, stimulation therapy may be delivered to achieve a therapy outcome and/or usage meeting a criteria per the examples of at least FIGS. 1A-9A.

In some examples the stimulation portion 2200 comprises an open loop parameter (e.g. 2222 in FIG. 8A) by which stimulation therapy (e.g. “use”) is applied without a feedback loop of sensed physiologic information. In some such examples, in an open loop mode the stimulation therapy is applied during a treatment period without (e.g. independent of) information sensed regarding the patient's sleep quality, sleep state, respiratory phase, AHI, etc. In some such examples, in an open loop mode the stimulation therapy is applied during a treatment period without (i.e. independent of) particular knowledge of the patient's respiratory cycle information.

In some examples the stimulation portion 2200 comprises a nightly titration parameter 2224 by which an intensity of stimulation therapy can be titrated (i.e. adjusted) to be more intense (e.g. higher amplitude, greater frequency, and/or greater pulse width) or to be less intense within a nightly treatment period. However, it will be understood that the previously described examples in association with at least FIGS. 1A-8 (and 9A-9E) may be performed without (e.g. independent of) a nightly titration parameter 2224 and instead be based on titration according to a time period parameter (430 in FIG. 6A) of more than a day, such as supra-day time period. Accordingly, in some examples, guiding therapy per a patient metric in examples of the present disclosure may be implemented solely according to time period (430 in FIG. 6A) of more than a nightly treatment period.

In some such examples, the nightly titration parameter 2224 may be implemented according to at least some aspects of the example methods and/or example devices of FIGS. 1A-8, for example, whether during initial stages (e.g. Stage 1 in FIG. 3) or later steady-state stages (e.g. Stage 2 in FIG. 3) of implementing guided therapy according to a patient metric. Accordingly, in some examples, the titration parameter may be implemented as automatic titration (e.g. 432) while in some examples, the titration parameter may be implemented via manual titration (436) by a patient (or clinician). In some examples, the titration parameter may be implemented via combination of patient/manual titration and automatic titration to guide the patient in a manner complementary with their manual titration.

In some such examples and as previously described, such titration may be implemented at least partially based on sleep quality, which may be obtained via sensed physiologic information, in some examples. It will be understood that such examples may be employed with synchronizing stimulation to sensed respiratory information (i.e. closed loop stimulation) or may be employed without synchronizing stimulation to sensed respiratory information (i.e. open loop stimulation).

In some examples, at least some aspects of the titration parameter 2224 of the stimulation portion 2200 and/or at least some aspects of titration as generally disclosed throughout FIGS. 1-9E in examples of the present disclosure may comprise (and/or may be implemented) in a manner complementary with and/or via at least some of substantially the same features and attributes as described in U.S. Pat. No. 8,938,299 to Christopherson et al., issued Jan. 20, 2015, entitled SYSTEM FOR TREATING SLEEP DISORDERED BREATHING, and which is hereby incorporated by reference in its entirety.

The sensing may include sensing of a particular target tissue generally and/or for a particular parameter, such as first respiration parameter 2305 and/or other physiologic parameter 2306 (FIG. 8B) which may or may not relate to respiration. FIG. 8B is a diagram 2300 illustrating an example arrangement of different target tissue(s) 2310 for sensing and/or target tissue(s) 2330 for stimulating.

In some examples, at least one of the target tissues 2310 may be used to sense a signal that generally corresponds to respiration to thereby provide information about a first respiration parameter 2305. The signal may be sensed from one of the target tissues 2310, on one or both lateral sides of the patient, and/or using a combination of the target tissues 2310. In some examples, one of the target tissues 2310 may be the first target tissue used to sense a first neural signal (and/or muscle signal), and a second target tissue nerve may be used if the first neural signal (and/or muscle signal) cannot be used (e.g., is no longer sensed, is noisy or other issues).

In some examples, the target tissues 2310 to be sensed may comprise an infrahyoid muscle (IHM)-innervating nerve 2312A, an IHM 2313A, a hypoglossal (HG) nerve 2314A, a genioglossus muscle 2315A, an internal superior laryngeal (iSL) nerve 2316A, a glossopharyngeal nerve 2317A, a phrenic nerve 2318A, a diaphragm muscle 2319A, and/or other nerves/muscles 2320A.

Meanwhile, the target tissues 2330 to be stimulated may comprise an IHM-innervating nerve 2312B, an IHM 2313B, an HG nerve 2314B, a genioglossus muscle 2315B, an iSL nerve 2316B, a glossopharyngeal nerve 2317B, a phrenic nerve 2318B, a diaphragm muscle 2319B, and/or other nerves/muscles 2320B.

In some examples, at least one of the target tissues 2330 may be stimulated. In some such examples, the stimulation is based on the sensed first respiration parameter 2305 and/or sensed other physiologic parameter. As previously described, any one of the respective target tissues 2310 may additionally serve as the target tissue(s) 2330 to be stimulated, in some examples. In some examples, multiple (e.g., at least two) of the target tissues 2330 may be stimulated. The stimulation of the multiple target tissues 2330 may occur simultaneously and/or sequentially. In some examples, such as those described above, the stimulation and sensing of the target tissues 2330 may be timed, such that sensing occurs at different times than stimulation. For example, a first target tissue may be sensed for a first plurality of sensing cycles to determine the first respiration parameter 2305 and then second target tissue may stimulation for a second plurality of stimulation cycles.

The timing, duration, amplitude, and/or selection of the target tissues 2330 to be stimulated may be set based on the signal (e.g., neural or muscle) sensed from at least one of the target tissues 2310. As a specific, and non-limiting example, the iSL nerve 2316A may be used to sense the first respiratory parameter and the iSL nerve 2316B (same or different portion) may be stimulated to elicit (via the CNS) the previously described reflex opening response that activates at least some of the target tissues 2330, such as (but not limited to) the HG nerve 2314B, the IHM-53 innervating nerve 2312B, which in turn causes activation (e.g., contraction) of their innervated muscles (e.g., upper airway dilators, such as the IHM 2313B and genioglossus muscle 2315B).

In some examples, a neural signal sensed from the iSL nerve 2316A may indicate an upper airway obstruction is occurring and/or continues after stimulating the iSL nerve 2316A. For example, for some patients, stimulating the iSL nerve 2316B to cause the reflex opening response may not be effective in increasing upper airway patency to a sufficient degree to ameliorate obstructive sleep apnea. In response, additional target tissue 2330 may be stimulated. For example, both the iSL nerve 2316B and other tissue, such as the IHM-innervating nerve 2312B or IHM 2313B, may be stimulated. In some such examples, other information indicative of a disease burden (e.g., AHI) may additionally or alternatively indicate to stimulate the additional target tissue(s) 2330.

It will be understood that some nerves/muscles may be considered to be upper airway patency-related tissue (e.g., nerves/muscles) in that direct sensing and/or direct stimulation of such nerves/muscles may have a direct effect on upper airway patency. For instance, stimulation of the HG nerve 2314B may cause protrusion of the tongue (via activation of the genioglossus muscle), which directly maintains and/or increases patency of the upper airway. Similarly, stimulation of the IHM-innervating nerve 2312B may cause (via activation of the sternothyroid muscle and/or other infrahyoid strap muscles), which may directly maintain and/or increase patency of the upper airway.

In some examples, stimulation of some target tissues 2330, such as the iSL nerve 2316B and/or afferent nerve fibers/branch of the glossopharyngeal nerve 2317B, may have an indirect effect, such as eliciting (via the CNS) a reflex opening response, which activates at least multiple upper airway dilator nerves/muscles. Such nerves are sometimes herein referred to as upper airway reflex-related sensory nerves. For instance, stimulation of afferent nerve fibers of the iSL nerve (and/or afferent nerve fibers/branch of the glossopharyngeal nerve) associated with mechanoreceptors in/near the upper airway may elicit (via the CNS) a reflex opening response to maintain and/or increase upper airway patency.

Meanwhile, in some examples, some target tissues may be used to affect respiration in other ways and/or more generally. For instance, an immediate effect of stimulation of the phrenic nerve 2318A includes activation of the diaphragm muscle 2319A, whose contraction induces a negative pressure within the lungs, thereby resulting in inspiration of air (passing through the upper airway) and other structures.

It will be understood that some example devices and/or some example methods may engage the phrenic nerve solely for stimulation to treat various types of apnea (e.g., central, mixed, other). However, some example devices and/or some example methods may engage the phrenic nerve solely for sensing or may engage the phrenic nerve for both sensing and stimulation.

Referring now in addition to FIG. 9A, which is a block diagram schematically representing an example care engine 2900. In some examples, the care engine 2900 may form part of a control portion 3000, as later described in association with at least FIG. 9B, such as but not limited to comprising at least part of the instructions 3011 and/or information 3012. In some examples, the care engine 2900 may be used to implement at least some of the various example devices and/or example methods of the present disclosure as previously described. In some examples, the care engine 2900 (FIG. 9A) and/or control portion 3000 (FIG. 9B) may form part of, and/or be in communication with, an implanted medical device 3060 (FIG. 9E). In some examples, the care engine 2900 (FIG. 9A) may be form part of, or be in communication with, one or more of the devices (e.g. 3060, 3070, 3074, 3076, 3080) in the arrangement of FIG. 9E.

As shown in FIG. 9A, in some examples the care engine 2900 comprises an input engine 2910 to receive and manage, process, and/or direct inputs such as, but not limited to, any or all of inputs of input portion 401 in the example device of FIG. 6A.

In some examples, the care engine 2900 comprises a sensing engine 2912 configured to sense one or more inputs (e.g., any of inputs in input portion 401 in FIG. 6A). In one aspect, at least the sensing engine 2912 of care engine 2900 in FIG. 9A directs the sensing of information, and/or receives, tracks, and/or evaluates sensed information obtained via one or more of the sensors (see sensors 2000 in FIG. 7) with care engine 2900 employing such information to determine a stimulation solution setting, for example, in a manner complementary with the examples of at least FIGS. 1-8 and 9B-9E.

In some examples, the care engine 2900 includes a notification engine 2914 configured to send and receive one or more notifications to one or more persons, which may include the patient and/or the clinician. In some examples, notifications can comprise any of the sort disclosed herein, such as instructions regarding inputs received, instructions for stimulation settings, the discovery of a solution stimulation setting, etc.

As further shown in FIG. 9A, in some examples the care engine 2900 comprises a patient metric engine 2916 to direct sensing of, and/or receive, track, evaluate, etc. parameters/patient metrics particularly associated with methods of the disclosure. In some examples, at least some of these patient metrics may comprise an example implementation, and/or at least some of substantially the same features and attributes as, a therapy outcome parameter as described in association with at least FIGS. 3 and 6. In some examples, the parameters may be associated with sleep disordered breathing (SDB) care. In some examples, as at least one example therapy outcome parameter, the patient metric engine 2916 comprises an AHI parameter to sense and/or track apnea-hypopnea index (AHI) information, which may be indicative of the patient's sleep quality among other therapy outcomes. In some examples, the AHI information is obtained via a sensing element, such as one or more of the various sensing types, modalities, etc., which may be implemented as described in various examples of the present disclosure.

As further shown in FIG. 9A, in some examples care engine 2900 comprises a stimulation engine 2918 to control stimulation of target tissues to treat various physiologic conditions, disease burdens, etc. In some examples, the target tissues may include respiratory-related tissues such as, but not limited to, an upper airway patency-related tissues (e.g. nerve, muscle, and/or combination thereof) to treat sleep disordered breathing (SDB). In some examples, the stimulation engine 2900 may implement, track, and/or control at least some of the aspects of the stimulation generator 424 in FIG. 6A, the stimulation portion 2200 in FIG. 8A, and/or other aspects of stimulation described throughout the examples of the present disclosure.

Moreover, with further reference to FIG. 9A, in some examples the above-mentioned electrocardiogram (ECG), ballistocardiograph sensing (BCG), seismocardiograph sensing (SCG), and/or accelerocardiograph sensing (ACG) may be employed in combination with the sensing of acceleration-based inclination angles (based on rotational movement of the rib cage during breathing), as noted in association with at least sensing engine 2912 of care engine 2900 and/or sensing portion 2000 of FIG. 7. In one aspect, the ECG, SCG, BCG, and/or ACG sensing may be used to perform sensing of Respiratory Sinus Arrhythmia (RSA) and by which respiration detection may be performed. In some such examples, the sensed RSA may be used to identify an inspiratory phase, expiratory active phase, and/or expiratory pause phase of a respiratory cycle and/or may be used to distinguish the respective phases from each other. In some such examples, such identifying and/or such distinguishing may be performed via the identifying an R-R interval to determine the sensed RSA, in which the R-R interval is shorter during inspiration and the R-R interval is faster during expiration.

It will be understood that the care engine 2900 may be implemented more generally in association with the various diseases associated with the disease burden indicators in addition to (or other than) sleep disordered breathing. In some such examples, the stimulation engine 2918 may more generally represent a therapy application engine, while the patient metric engine 2916 more generally represent a disease burden indication parameters engine, and the respiration engine 2912 may more generally represent at least one physiologic parameter primarily associated with the particular disease.

FIG. 9B is a block diagram schematically representing an example control portion 3000. In some examples, control portion 3000 provides one example implementation of a control portion forming a part of, implementing, and/or generally managing sensors, sensing element(s), stimulation elements, power/control elements (e.g. pulse generator), data models, devices, user interfaces, instructions, information, engines, elements, functions, actions, and/or methods, as described throughout examples of the present disclosure.

In some examples, control portion 3000 includes a controller 3002 and a memory 3010. In general terms, controller 3002 of control portion 3000 comprises at least one processor 3004 and associated memories. The controller 3002 is electrically couplable to, and in communication with, memory 3010 to generate control signals to direct operation of at least some of the sensors, sensing element(s) stimulation elements, power/control elements (e.g. pulse generators), devices, user interfaces, instructions, information, engines, elements, functions, actions, and/or methods, etc. as described throughout examples of the present disclosure. In some examples, these generated control signals include, but are not limited to, employing instructions 3011 and/or information 3012 stored in memory 3010 to at least discovering a solution stimulation setting as outlined, for example, in FIGS. 1A and/or 3. Such discovery of a solution stimulation setting may comprise part of identifying sleep disordered breathing (SDB) and directing and managing treatment of sleep disordered breathing such as obstructive sleep apnea, hypopnea, and/or central sleep apnea. In some instances, the controller 3002 or control portion 3000 may sometimes be referred to as being programmed to perform the above-identified actions, functions, etc. such that the controller 3002, control portion 3000 and any associated processors may sometimes be referred to as being a special purpose computer, control portion, controller, or processor. In some examples, at least some of the stored instructions 3011 are implemented as, or may be referred to as, a care engine, a sensing engine, patient metric engine, input engine, notification engine and/or stimulation engine. In some examples, at least some of the stored instructions 3011 and/or information 3012 may form at least part of, and/or, may be referred to as a care engine, sensing engine, patient metric engine, input engine, notification engine and/or stimulation engine.

In response to or based upon commands received via a user interface (e.g. user interface 3040 in FIG. 9D) and/or via machine readable instructions, controller 3002 generates control signals as described above in accordance with at least some of the examples of the present disclosure. In some examples, controller 3002 is embodied in a general purpose computing device while in some examples, controller 3002 is incorporated into or associated with at least some of the sensors, sensing element, stimulation elements, power/control elements (e.g. pulse generators), devices, user interfaces, instructions, information, engines, functions, actions, and/or method, etc. as described throughout examples of the present disclosure.

For purposes of this application, in reference to the controller 9802, the term “processor” shall mean a presently developed or future developed processor (or processing resources) that executes machine readable instructions contained in a memory. In some examples, execution of the machine readable instructions, such as those provided via memory 3010 of control portion 3000 cause the processor to perform the above-identified actions, such as operating controller 3002 to implement the sensing, monitoring, discovering a solution stimulation setting, stimulation, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 3010. In some examples, the machine readable instructions may comprise a sequence of instructions, a processor-executable machine learning model, or the like. In some examples, memory 3010 comprises a computer readable tangible medium providing non-volatile storage of the machine readable instructions executable by a process of controller 3002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 3002 may be embodied as part of at least one application-specific integrated circuit (ASIC), at least one field-programmable gate array (FPGA), and/or the like. In at least some examples, the controller 3002 is not limited to any specific combination of hardware circuitry and machine readable instructions, nor limited to any particular source for the machine readable instructions executed by the controller 3002.

In some examples, control portion 3000 may be entirely implemented within or by a stand-alone device.

In some examples, the control portion 3000 may be partially implemented in one of the sensors, sensing element, monitoring devices, treatment devices (e.g. stimulation devices, or portions thereof), etc. and partially implemented in a computing resource (e.g. at least one external resource) separate from, and independent of, the treatment devices (or portions thereof) but in communication with the treatment devices (or portions thereof). For instance, in some examples control portion 3000 may be implemented via a server accessible via the cloud and/or other network pathways. In some examples, the control portion 3000 may be distributed or apportioned among multiple devices or resources such as among a server, a treatment device (or portion thereof), and/or a user interface.

In some examples, control portion 3000 includes, and/or is in communication with, a user interface 3040 as shown in FIG. 9D.

FIG. 9C is a diagram schematically illustrating at least some example arrangements of a control portion 3020 by which the control portion 3000 (FIG. 9B) can be implemented, according to one example of the present disclosure. In some examples, control portion 3020 is entirely implemented within or by a pulse generator (IPG) 3025, which has at least some of substantially the same features and attributes as a pulse generator (e.g. power/control element) as previously described throughout the present disclosure. In some examples, control portion 3020 is entirely implemented within or by a remote control 3030 (e.g. a programmer) external to the patient's body, such as a patient control 3032 and/or a physician control 3034. In some examples, the control portion 3000 is partially implemented in the IPG 3025 and partially implemented in the remote control 3030 (at least one of patient control 3032 and physician control 3034). In some examples, the control portion 3000 is at least partially implemented via a clinician portal 3036, which may or may not be in complementary relation with elements 3025 and 3030.

FIG. 9D is a block diagram schematically representing user interface 3040, according to one example of the present disclosure. In some examples, user interface 3040 forms part or and/or is accessible via a device external to the patient and by which the therapy system may be at least partially controlled and/or monitored. The external device which hosts user interface 3040 may be a patient remote (e.g. 3032 in FIG. 9C), a physician remote (e.g. 3034 in FIG. 9C) and/or a clinician portal (e.g. 3036 in FIG. 9C). In some examples, user interface 3040 comprises a user interface or other display that provides for the simultaneous display, activation, and/or operation of at least some of the sensors, sensing element, stimulation elements, power/control elements (e.g. pulse generators), devices, user interfaces, instructions, information, engines, functions, actions, and/or method, etc. In some examples, at least some portions or aspects of the user interface 3040 are provided via a graphical user interface (GUI), and may comprise a display 3044 and input 3042.

FIG. 9E is a block diagram 3050 which schematically represents some example implementations by which a medical device (MD) 3060 (which may, in some examples, include one or more stimulation elements 47), a sensing element (e.g. a monitor in some examples), and the like may communicate wirelessly with devices outside the patient. In some examples, the medical device 3060 may comprise at least some implantable components (hence in some examples may sometimes be referred to as an implantable medical device) and/or at least some external components. Similarly, the sensing element (associated with the medical device 3060) may comprise at least some implantable components (hence in some examples may sometimes be referred to as an implantable sensing element) and/or at least some external components.

As shown in FIG. 9E, in some examples, the MD 3060 may communicate with at least one of patient app 3072 on a mobile device 3070, a patient remote control 3074, a clinician programmer 3076, and a patient management tool 3080. The patient management tool 3080 may be implemented via a cloud-based portal 3082, the patient app 3072, and/or the patient remote control 3074, each of which may comprise a user interface (e.g. having a display, input) such as user interface 3040 in FIG. 9D.

Among other types of data, these communication arrangements enable the MD 3060 to communicate, display, manage, etc. the AHI determination information, ODI determination information, as well as to allow for adjustment to the various elements, portions, etc. of the example devices and methods if and where desired. In some examples, the various forms of identified sleep disordered breathing (e.g. AHI, ODI) (and/or other information available via various examples of the present disclosure) may be displayed to a patient and/or clinician via one of the above-described external devices. The displayed information may comprise each event of sleep disordered breathing, a nightly aggregate of such events, or trends regarding such sleep disordered breathing.

With this in mind, FIG. 9F is a flow diagram illustrating an example method 3100 for managing titration. In some examples, the method comprises participation by a patient implanted with an IMD. In some examples, the method 3100 may comprise an example implementation of, and/or at least some of substantially the same features as, the examples of FIGS. 1A-9E. In some example implementations, the various actions of the method may be implemented via a control portion (e.g. 3000, 3020 in FIGS. 9B-9C) programmed to, and/or via machine readable instructions stored in non-volatile memory and executable via a processor to, perform the actions of method 3100.

With further reference to FIG. 9F, as shown at 3102, method 3100 may comprise receiving data relating to stimulation delivered by a medical device, such as (but not limited to) device 3060 in FIG. 9E, pulse generator 3025 in FIGS. 9C, 424 in FIG. 6A, stimulation element 47, and/or external 70 in FIG. 1B. In some examples, the data may be received at a first device which includes a user interface such as (but not limited to) user interface 3040 in FIG. 9C. In some examples, the first device may comprise, and/or sometimes be referred to as, a management device.

In some such examples, in method 3100 the first device to receive data (at 3102) may comprise a patient control (e.g. 438 in FIGS. 6A, 3032 in FIGS. 9C, 3074 in FIG. 9E), an app on a mobile device (e.g. 482/480 in FIGS. 6A, 3072/3070 in FIG. 9E, etc.), an app on a portal (e.g. clinician or patient, such as 486/484 in FIGS. 6A, 3082/3080 in FIG. 9E), The first device may receive the data upon various occasions in which the receiving device is in communication with the “stimulating” medical device.

In some examples, the first device may display titration data among other information related to stimulation therapy, sensing, and/or other functions. In some examples, the displayed information may comprise at least some of substantially the same information illustrated in at least FIGS. 2, 4-5, 6B-6E and/or information derived from these FIGS. The information may be displayed for a patient and/or for a clinician.

As shown at 3104, in some examples method 3100 comprises receiving input, via the user interface (e.g. display, input), from a user (e.g. patient and/or clinician) to select and/or modify a parameter of stimulation therapy, sensing parameters, and/or a combination thereof.

As shown at 3106, in some examples method 3100 may comprise, based on the parameter selection (at 3104), determining a titration schedule and transmitting the titration schedule to the medical device (e.g. for delivering stimulation therapy). The titration schedule may be viewed on the display of the first device by the patient (and/or a clinician).

In some examples, the method 3100 comprises authorizing the patient to independently make modifications to the titration schedule. In some such examples, a clinician can review and approve the “independent” parameter selection by the patient before the titration schedule is transmitted to the implanted medical device (e.g. for delivering stimulation therapy).

In some examples, the patient modifications are limited within a pre-approved range and modifications outside of the pre-approved range are reviewed and approved before the titration schedule is transmitted to the medical device.

As shown at 3108, in some examples, method 3100 may further comprise sending instructions from the first device (or other source) to the medical device to deliver stimulation based on the updated titration schedule and/or based on updated sensing parameters. In some such examples, the method 3100 may comprise delivering the stimulation via the medical device to the target tissue of the patient based on the updated titration schedule, updated sensing parameters, and/or other parameters.

As shown in FIG. 9G, in some examples, a method 3200 comprises managing a patient titration (e.g. two or more) with regard to stimulation therapy, sensing, and/or other parameters. In some examples, the method 3200 may comprise at least some of substantially the same features as, and/or an example implementation of, method 3100, wherein the managing titration may be implemented for at least two patients, in some examples.

In some examples, as shown at 3203 method 3200 comprises displaying, on the user interface, a status of titration for each patient. In some such examples, the displaying comprises displaying a priority for the patients based on the status of titration for each patient. Displaying the priority between the patients (e.g. which patient(s) have the highest priority) may comprise displaying a more conspicuous visual indicator for the highest priority patients.

As shown at 3205, in some examples method 3200 comprises implementing the titration for each patient according to a titration schedule, which in some examples may comprise incrementally adjusting the stimulation parameters (e.g. intensity parameters such as amplitude and/or other parameters) until reaching a final set of stimulation parameters. In some examples, the incremental adjustments may comprise incrementally increasing the stimulation intensity (and/or other parameters) and/or decrementally decreasing the stimulation intensity (and/or other parameters).

As shown at 3207, in some examples method 3200 comprises displaying the status of progression of the titration for each patent and displaying an updated priority for the patients based on their titration progression status. In some such examples, the method 3200 may comprise displaying information (e.g. parameters) to assist the patient with any titration adjustments (e.g. increases or decreases) of stimulation intensity initiated by the patient.

In some examples, displaying (e.g. via the user interface) a relative priority between multiple patients may assist the clinician in determining which patients may need help the soonest in selecting and/or implementing modifications to stimulation intensity (and/or other parameters) as part of their titration.

In some examples, method 3200 may comprise displaying updates to the priority of the patients based on updates to the titration progression status of each respective patient.

As shown at 3207, in some examples method 3200 may comprise sending instructions (e.g. from the first device to the medical device (e.g. including at least an implanted stimulation element)) for, and/or implementing, delivery of stimulation based on the updated titration schedule and/or based on updated sensing parameters. In some such examples, the method 3200 may comprise delivering the stimulation to the target tissue of the patient based on the updated titration schedule, updated sensing parameters, and/or other parameters.

As previously noted, determining sleep disordered breathing and/or treating sleep disordered breathing provides just one example of managing disease for a patient, such that a sleep disordered breathing indicator (e.g. AHI) may comprise just one example of a disease burden indicator. At least some further examples are provided below.

In one example, a method for treating a medical condition (e.g., sleep disordered breathing (SDB)) may include: receiving, for each of a multiple number of sleep treatment periods 430, at least one selected stimulation therapy amplitude 442 applied to an upper airway patency-related nerve (via 460a, 460b, for example); wherein the values of at least two of the selected stimulation therapy amplitudes are different values. The example method may further include receiving data indicative of a therapy outcome 412 and of a usage 418 for each of the sleep treatment periods. The example method may further include assessing the data 426 indicative of the at least one sleep disordered breathing therapy outcome for each nightly sleep treatment period in relation to the data indicative of the first parameter over the selective multiple number of nightly sleep treatment periods as a function of the selected stimulation therapy amplitude; and based on the assessment, identifying a target therapy amplitude or solution stimulation setting to maintain, reduce or increase the stimulation setting 440.

Yet another example method of treating a medical condition, such as sleep disordered breathing, includes applying, via an implanted pulse generator (e.g., 425, 3025), electrical stimulation therapy to at least one upper airway patency-related nerve at a first therapy amplitude 442 for a first treatment interval 434 including at least one nightly sleep treatment period 430. The example method may further include recording, for the first treatment interval and via a patient device 438, 480, information including a value of the first therapy amplitude during the first treatment interval (see also, FIGS. 2, 4, or 5, for example). The example method may further include recording data indicative of the at least one sleep disordered breathing therapy outcome 412 for each nightly sleep treatment period of the first treatment interval and sending the recorded data of the at least one sleep disordered breathing therapy outcome 412 to the clinician programmer 484. The example method can further include applying, via the implanted pulse generator 424, electrical stimulation therapy to at least one upper airway patency-related nerve at a second therapy amplitude that is different than the first therapy amplitude for a second treatment interval including at least one nightly sleep treatment period. The example method may further include recording, for the second treatment interval and via the patient device (438/480, for example), information including values of the therapy amplitude 442 during the second treatment interval. The example method may further include recording data indicative of the at least one sleep disordered breathing therapy outcome 412 for each nightly sleep treatment period of the second treatment interval and sending the recorded data to the clinician programmer 484. The example method may further include assessing the data 412 indicative of the at least one sleep disordered breathing therapy outcome for each nightly sleep treatment period of the first treatment interval at the first therapy amplitude in comparison to the data indicative of the at least one sleep disordered breathing therapy outcome for each nightly sleep treatment period of the second treatment interval at the second therapy amplitude. The method may further include, identifying, based on the assessment 426, a solution stimulation setting. Subsequent steps, in some examples, may include maintaining, reducing or increasing the stimulation therapy setting 440 to be set to the solution stimulation setting. All of the aforementioned recording can be stored in memory 3010, for example, as illustrated in FIG. 9B.

In one example, a method comprises guiding stimulation therapy for a patient metric. In some examples, the method comprises implementing the stimulation therapy via delivering a stimulation signal, via a stimulation element, to a target tissue. In some examples, the target tissue can comprise an upper airway patency-related tissue. In some examples, the upper airway patency-related tissue can comprise at least one of a hypoglossal nerve and an infrahyoid muscle-related nerve. In some examples, guiding the stimulation therapy can comprise tracking patient-initiated changes of the stimulation therapy, evaluation of a therapy outcome, or automatically titrating in selecting at least one stimulation energy parameter. In some examples, a single stimulation energy parameter is varied in the step of automatically titrating. In some examples, the stimulation energy parameter that is varied can be changed. In some examples, a solution stimulation energy parameter value is determined. In some examples, after automatic determination of a solution stimulation energy parameter value, at least one of communicating a notification to a patient and/or requesting confirmation from the patient occurs. In some examples, guiding the stimulation therapy comprises tracking values of at least one patient metric over a supra-day time window and, based on tracked values, incrementing or decrementing a stimulation energy value of the stimulation therapy. In some examples, guiding comprises initiating the guiding from an initial therapy point to discover a solution stimulation setting at which criteria for the patient metric is met. In some examples, the solution stimulation setting comprises a range of stimulation settings. In some examples, the initial therapy point is first post-implant use of therapy device. In some examples, discovering the solution stimulation setting comprises, in a first stage, progressively increasing the stimulation setting of the stimulation therapy while maintaining other stimulation therapy parameters as fixed parameters to discover the solution stimulation setting. In some examples, discovering the solution stimulation setting comprises, in a first stage, varying one single stimulation setting of the stimulation therapy at a time for a plurality of consecutive nightly treatment periods, to look for spot until the solution stimulation setting is discovered. In some examples, the guiding comprises, from a first therapy point, at a plurality of intervals, changing a value of the stimulation therapy; and evaluating the patient metric.

In some examples, the patient metric comprises both therapy outcome and usage. In some examples, the therapy outcome comprises AHI and criteria is met for discovering solution stimulation settings when a plurality of consecutive decreases in AHI at a prior stimulation energy setting is tracked followed by at least one increase in AHI after an increase in stimulation therapy energy setting. In some examples, discovering a solution stimulation setting includes identifying a reduction in usage associated with an increase in the stimulation therapy energy setting. In some examples, the reduction is observed over a plurality of nightly treatment periods. In some examples, the plurality of intervals comprise a weekly time period. In some examples, the patient metric is implemented to comprise both a therapy outcome parameter and an usage parameter. In some examples, the guiding is implemented over a supra-day time period. In some examples, the supra-day time period comprises multiple number of nightly sleep treatment periods. In some examples, the supra-day time period comprises at least 30 days. In some examples, the supra-day time period comprises no less than 60 days.

In one example method of treating sleep disordered breathing of a patient, the method comprises tracking, from an initial patient therapy point of electrical stimulation of at least one upper airway patency-related tissue by a stimulation element, changes in stimulation energy values for the patient over a multiple number of sleep treatment periods toward a solution stimulation settings amplitude value at which criteria are met for a target therapy outcome parameter and a target usage parameter. In some examples, the criteria is defined at least in part by whether usage of the stimulation element to deliver stimulation at the stimulation amplitude values is maintained or does not decrease below a threshold. In some examples, the stimulation element comprises an implantable pulse generator and a stimulation lead/electrode connected to an implantable pulse generator. In some examples, the initial therapy point is a first, post-implant actual therapy stimulation amplitude. In some examples, the therapy outcome parameter includes at least one of an average apnea frequency per sleep treatment period; ESS, snoring; arousals; and patient perceived sleep quality. In some examples, the target therapy outcome parameter comprises a deviation from a threshold in average apnea frequency per sleep treatment period. In some examples, the criteria includes when the target therapy outcome parameter is maintained or is not below a threshold. In some examples, the criteria for the target therapy outcome parameter includes a threshold maximum decrease in average apnea frequency per sleep treatment period from a baseline average apnea frequency per sleep treatment period. In some examples, the target usage parameter includes a target percentage of deliverable therapy within a nightly treatment period. In some examples, the maximum usage is defined as a number of hours of stimulation therapy for each of the multiple number of sleep treatment periods. In some examples, the number of hours is at least four. In some examples, the target usage parameter comprises a number of nights of the multiple number of sleep treatment periods for which a minimum number hours of stimulation therapy was delivered. In some examples, stimulation energy includes one or more of amplitude, pulse width and pulse rate. In some examples, the change in stimulation energy is initiated by the patient. In some examples, the change in stimulation energy is automatically initiated. In some examples, the criteria is defined at least in part by whether the target therapy outcome parameter is within a range. In some examples, the method comprises implementing the stimulation therapy via delivering a stimulation signal, via a stimulation element, to a target tissue.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A device comprising:
a control portion programmed to guide delivery of electrical stimulation over a supra-day time period, via a stimulation element in stimulating relation to an upper airway patency-related tissue, according to a patient metric.

2. The device of claim 1, wherein the upper airway patency-related tissue comprises at least one of a hypoglossal nerve and an infrahyoid muscle-innervating nerve.

3. The device of claim 1, wherein the control portion is configured to guide delivery of electrical stimulation by at least one of:
tracking patient-initiated changes of the electrical stimulation;
determining a solution stimulation energy parameter value;
evaluating a therapy outcome; or automatically titrating in selecting at least one stimulation energy parameter.

4. The device of claim 3, wherein a single stimulation energy parameter is varied in automatically titrating.

5. The device of claim 4, wherein the single stimulation energy parameter is changed.

6. The device of claim 3, wherein the control portion is configured to communicate a notification to a patient and/or request confirmation from the patient of the solution stimulation energy parameter value.

7. The device of claim 1, wherein the control portion is configured to guide delivery of electrical stimulation by tracking values of at least one patient metric over a supra-day time period and, based on tracked values, incrementing or decrementing a stimulation energy value of the stimulation therapy.

8. The device of claim 1, wherein the control portion is configured to guide delivery of electrical stimulation by one of:
initiating guiding from an initial therapy point to discover a solution stimulation setting at which criteria for the patient metric is met; or
from a first therapy point, at a plurality of intervals, changing a value of the stimulation therapy, and evaluating the patient metric.

9. The device of claim 8, wherein the solution stimulation setting comprises a range of stimulation settings discovered by, in a first stage, one of:
progressively increasing the stimulation setting of the stimulation therapy while maintaining other stimulation therapy parameters as fixed parameters to discover the solution stimulation setting; or
varying one single stimulation setting of the stimulation therapy at a time for a plurality of consecutive treatment periods until the solution stimulation setting is discovered.

10. The device of claim 8, wherein the patient metric comprises both therapy outcome and usage.

11. The device of claim 10, wherein therapy outcome comprises AHI and criteria is met for discovering solution stimulation settings when a plurality of consecutive decreases in AHI at a prior stimulation energy setting is tracked followed by at least one increase in AHI after an increase in stimulation therapy energy setting.

12. The device of claim 10, wherein the solution stimulation setting is discovered by identifying a reduction in usage associated with an increase in the stimulation therapy energy setting.

13. (canceled)

14. The device of claim 1, wherein the supra-day time period comprises at least 30 days.

15. The device of claim 1, wherein the control portion is configured to track, from an initial patient therapy point of electrical stimulation of the upper airway patency-related tissue by the stimulation element, changes in stimulation energy values for a patient over the supra-day time period toward a solution stimulation setting energy value at which criteria are met for:
a target therapy outcome parameter; and
a target usage parameter,
wherein the supra-day time period comprises a multiple number of sleep treatment periods.

16. The device of claim 15, wherein the solution stimulation energy value is an amplitude value.

17. The device of claim 1, wherein the patient metric comprises a therapy outcome including at least one of:
average apnea frequency per sleep treatment periods (AHI);
Epworth Sleepiness Scale (ESS);
snoring:
arousals; and
patient perceived sleep quality.

18. The device of claim 1, wherein the control portion is configured to track changes in stimulation energy values for a patient over the supra-day time period toward a solution stimulation setting at which criteria are met for a target therapy outcome parameter and a target usage parameter, wherein stimulation energy includes one or more of amplitude, pulse width, and pulse rate and wherein the supra-day time period comprises a multiple number o tment periods.

19. The device of claim 1, wherein the stimulation element comprises an implantable stimulation electrode arrangement and a pulse generator in communication with the implantable stimulation electrode arrangement.

20. The device of claim 19, wherein the pulse generator comprises an implantable pulse generator sized and shaped for implantation in a head-and-neck region of a patient;

21. The device of claim 20, further comprising a lead connecting the implantable pulse generator to the implantable stimulation electrode arrangement.
